(19) United States
(12) Patent Application Publication
(10) Pub. No.: US 2013/0157293 A1
(43) Pub. Date: Jun. 20, 2013

(54) PHARMACEUTICAL MANUFACTURING METHODS

(71) Applicants: MARK E BRANUM, Bellevue, WA (US); Maralee McVean, Seattle, WA (US); Just Justesen, Risskov (DK); Eric J. Tarcha, Seattle, WA (US)

(72) Inventors: MARK E BRANUM, Bellevue, WA (US); Maralee McVean, Seattle, WA (US); Just Justesen, Risskov (DK); Eric J. Tarcha, Seattle, WA (US)

(73) Assignee: Kineta Two, LLC, Seattle, WA (US)

(21) Appl. No.: 13/605,858

(22) Filed: Sep. 6, 2012

Related U.S. Application Data
(63) Continuation of application No. 11/831,368, filed on Jul. 31, 2007, now abandoned.
(60) Provisional application No. 60/835,078, filed on Aug. 1, 2006.

(51) Int. Cl. C12N 9/00

(52) U.S. Cl. CPC C12N 9/93
USPC 435/7.92; 435/183; 435/29

(57) ABSTRACT

The invention describes methods for manufacturing oligoadenylate synthetase (OAS) proteins for use as active pharmaceutical ingredients in pharmaceutical compositions. A manufacturing method is described that produces large quantities of concentrated, highly active OAS protein for use in pharmaceutical compositions for the treatment of a variety of diseases including viral infection. Methods for monitoring and validating the manufacturing process are also described.

### TECHNICAL FIELD

The present invention relates to methods of manufacturing pharmaceutical compositions for the treatment of virus infections and cancer in mammals.

### BACKGROUND OF THE INVENTION

Oligoadenylate synthetase (OAS) proteins are interferon-induced proteins characterized by their capacity to catalyze the synthesis of 2-prime, 5-prime oligomers of adenosine (2-5As). OAS proteins mediate antiviral and pro-apoptotic activities in mammalian cells. We have previously demonstrated an association of mutations in members of the OAS gene family with resistance to virus infection in the human population. We have described methods of using the OAS genes and proteins as pharmaceutical compositions and have further described variant, modified and derivative forms of the OAS genes and proteins with improved drug properties.

Hovanessian et al., EMBO 6: 1273-1280 (1987) found that interferon-treated human cells contain several OAS isoforms corresponding to proteins of 40 (OAS1), 46 (OAS1), 69 (OAS2), and 100 (OAS3) kD. Marie et al., Biochem. Biophys. Res. Commun. 160:580-587 (1989) generated highly specific polyclonal antibodies against p69, the 69-kD OAS2. By screening an interferon-treated human cell expression library with the anti-p69 antibodies, Marie and Hovanessian, J. Biol. Chem. 267: 9933-9939 (1992) isolated a partial OAS2 cDNA. They screened additional libraries with the partial cDNA and recovered cDNAs encoding two OAS2 isoforms. The smaller isoform is encoded by two mRNAs that differ in the length of the 3-prime untranslated region.

Northern blot analysis revealed that OAS2 is expressed as four interferon-induced mRNAs in human cells. The predicted OAS2 proteins have a common 683-amino acid sequence and different 3-prime termini. According to SDS-PAGE of in vitro transcription/translation products, two isoforms have molecular masses of 69 and 71 kD. Both isoforms exhibited OAS activity in vitro. Sequence analysis indicated that OAS2 contains two OAS1-homologous domains separated by a proline-rich putative linker region. The N- and C-terminal domains are 41% and 53% identical to OAS1, respectively. Likewise, OAS3 contains three tandem units of the OAS1-homologous domains.

By fluorescence in situ hybridization and by inclusion within mapped clones, Hovnanian et al., Genomics 52: 267-277 (1998) determined that the OAS1, OAS2, and OAS3 genes are clustered with a 130-kb region on 12q24.2. 2-5As bind to and activate RNase L, which degrades viral and cellular RNAs, leading to inhibition of cellular protein synthesis and impairment of viral replication.

A fourth human OAS gene, referred to as OASL, differs from OAS1, OAS2 and OAS3 in that OASL lacks enzyme activity. The OASL gene encodes a two-domain protein composed of an OAS unit fused to a 164 amino acid C-terminal domain that is homologous to a tandem repeat of ubiquitin. (Eskildsen et al., Nuc. Acids Res. 31:3166-3173, 2003; Kakuta et al., J. Interferon & Cytokine Res. 22:981-993, 2002.)

The present invention fulfills needs in the art by providing methods for manufacturing oligoadenylate synthetase proteins for a variety of uses including in pharmaceutical compositions.

### BRIEF SUMMARY OF THE INVENTION

The present invention relates to methods for manufacturing oligoadenylate synthetase proteins such as mammalian or human oligoadenylate synthetase proteins.

The invention further relates to methods for testing the specific activity and antiviral potency of lots of manufactured OAS protein.

The invention further relates to methods of validating an OAS protein manufacturing process.

The invention further relates to the manufacture of drug products, active pharmaceutical ingredients and pharmaceutical compositions.

The invention relates to the manufacture of OAS proteins under Good Manufacturing Practices. In a still further embodiment, the invention relates to analytical methods used to monitor and validate the manufacture of OAS proteins under Good Manufacturing Practices.

The invention relates to the use of a nucleic acid encoding an OAS protein in a manufacturing process. In a further embodiment, the manufacturing process is a drug or active pharmaceutical ingredient manufacturing process.

In a still further embodiment, an OAS gene can be used to manufacture a drug product using recombinant DNA technologies. In a still further embodiment, expression of the OAS polypeptide can be effected in mammalian, insect, plant, bacterial or fungal cells or organisms. In a still further embodiment, a T7 bacteriophage promoter drives expression of the OAS gene, and the host E. coli strain expresses the T7 bacteriophage polymerase. In a still further embodiment, induction of recombinant OAS gene expression is effected by changes in culture temperature, carbon source, by the addition of natural or synthetic sugars, or by the addition of osmolytes.

The invention relates to methods of manufacturing OAS proteins involving both soluble and insoluble protein intermediates. The invention further relates to methods of manufacturing that favor either insoluble or soluble OAS protein intermediates. The invention further relates to methods of manufacturing OAS proteins involving the formation of inclusion bodies. In one embodiment, the inclusion bodies are formed in Escherichia coli (E. coli). The invention further relates to isolation of OAS protein-containing inclusion bodies from recombinant host organisms using one or more of the following methods: homogenization, centrifugation, hollow fiber filtration, membrane filtration, tangential flow filtration, and expanded bed absorption. In a still further embodiment, OAS protein containing inclusion bodies can be washed to increase purity using solutions containing one or more of the following: chelating agents, detergents, buffers, salts, chaotropic agents and the like.

In a still further embodiment, recombinant host organisms are lysed using one or more of: chemicals, detergents, enzymes, and mechanical methods. In a still further embodiment, enzymes include lysozymes, deoxyribonucleases, and ribonucleases.

The invention relates to methods of solubilizing and refolding OAS proteins. In one embodiment, solubilization and refolding are effected by the use of a chaotropic agent, a reducing agent or both. In a still further embodiment, solubilization and refolding are enhanced by the addition of one or more of the following solution components: salts, buffers, chelating agents, detergents, amine containing reagents, amino acids, polyols, polymers and chelating agents. In a still further embodiment, one or more of the following methods are used to enhance the solubilization and refolding of OAS proteins: change in temperature, low temperature, change in pH, high pressure, increased volume, dilution, and sonication.

The invention relates to methods of stabilizing and solubilizing OAS proteins involving the use of one or more of: chaotropic agents, surfactants, reducing agents, polyols, polymers, sugars, cyclodextrans, albumins, amino acids and the like. The invention further relates to methods of monitoring for the correct and complete refolding of OAS proteins. In one embodiment, refolding efficiency is measured by monitoring the oligoadenylate synthesizing capacity or specific activity of refolded OAS proteins. In a still further embodiment, refolding is measured using chromatographic methods, such as for example high performance liquid chromatography (HPLC) and fast protein liquid chromatography (FPLC).

The invention relates to methods of capturing, isolating, or purifying OAS proteins from complex biological matrices. In one embodiment, OAS proteins are captured, isolated or purified from mammalian or prokaryotic cells or from inclusion bodies. In a still further embodiment, OAS proteins are captured, isolated, or purified from prokaryotic or eukaryotic organisms or tissues. In a still further embodiment, capture, isolation, or purification is affected by the use of cation exchange chromatography. The invention is not limited by the type of cation exchange resin used. In a still further embodiment, initial capture, isolation, or purification is mediated by the use of affinity columns, such as for example those derivatized with deoxyribonucleic acid or ribonucleic acid. In a still further embodiment, nicotinamide dye columns are used. In a still further embodiment, heparin columns, which have both affinity and cation exchange properties, are used to capture, isolate, and purify OAS proteins. In a still further embodiment, carboxymethyl (CM) cation exchange resins are used.

The invention relates to methods for purifying or isolating OAS proteins involving the use of hydrophobic interaction chromatography. The invention is not limited by the type of hydrophobic interaction chromatography media used. In one specific embodiment, Phenyl HP columns are used to purify OAS proteins from contaminating proteins and from endotoxin or lipopolysaccharide.

The invention relates to methods for purifying or isolating OAS proteins involving the use of reverse phase HPLC or FPLC. The invention is not limited by the specific method of reverse phase HPLC or FPLC employed.

The invention relates to the use of chromatography methods for separating enzymatically active from enzymatically inactive forms of the OAS proteins.

The invention relates to methods of purifying or isolating OAS proteins involving the use of anion exchange chromatography. The invention is not limited by the type of media employed as an anion exchanger. In one specific embodiment, diethylaminoethyl (DEAE) sepharose is used for OAS protein purification. The invention relates to the use of anion exchange chromatography to purify OAS proteins from contaminating proteins, endotoxin, lipopolysaccharide, nucleic acids and pyrogens. The invention also relates to the use of anion exchange membranes and anion exchange cartridges distinct from traditional column chromatography. The invention is not limited by the type of anion exchange membrane, resin, or cartridge used.

The invention relates to the purification of OAS proteins from complex biological matrices by using affinity chromatography. In one embodiment, nickel-derivatized chromatography columns are used. In another example deoxyribonucleic acid and ribonucleic acid columns are used. In a still further example, the transgene from which OAS proteins are expressed contains an affinity tag such that a fusion protein is expressed which contains both the affinity tag and the OAS protein together in a single polypeptide. The invention is not limited by the type of affinity tag used, if any, and the type of affinity media used for purification.

The invention relates to the use of cation exchange chromatography, heparin column chromatography, tangential flow filtration, anion exchange chromatography, ultrafiltration, diafiltration, hydrophobic interaction chromatography, gel filtration, or affinity chromatography for purification, buffer exchange and for the concentration of solutions containing OAS proteins. The invention further relates to the use of a combination of these methods for buffer exchange and OAS protein concentration.

### BRIEF DESCRIPTION OF THE FIGURES

FIG. 1 is an overview of the OAS Manufacturing Process. The manufacturing process for OAS proteins can be divided into nine discrete steps, as described in this figure.

FIG. 2 is an example of several chromatograms generated by FPLC during steps 4-8 of the manufacturing process as described in FIG. 1.

FIG. 3 is an example of SDS-PAGE gel chromatographic analysis of samples generated during the manufacturing process showing increased levels of purity of OAS proteins as manufacturing steps are completed and the product is purified to homogeneity.

FIG. 4 and Table 1 are examples of the results of OAS enzyme activity assays (to measure specific activity) performed on in-process manufacturing samples and purified protein using the radioactive enzyme assay described in the specification.

FIG. 5 demonstrates the effect of different buffer conditions, reducing agents, pH, time, and temperature on refolding efficiency and subsequent specific activity of the manufactured OAS protein.

FIG. 6 shows an exemplary HPLC chromatogram of the NAD-dATP assay demonstrating the production of NAD-AMP from NAD and dATP by an OAS protein purified according to the methods of the specification.

FIG. 7 shows typical results obtained after assessing the antiviral potency of an OAS protein purified via the methods described in the specification.

FIG. 8 is a listing of polynucleotide sequences (SEQ ID NO: 1-8) that are useful in exemplary modes of practicing the present invention.

FIG. 9 is a listing of polypeptide sequences (SEQ ID NO: 9-16) that are produced in exemplary modes of practicing the present invention.

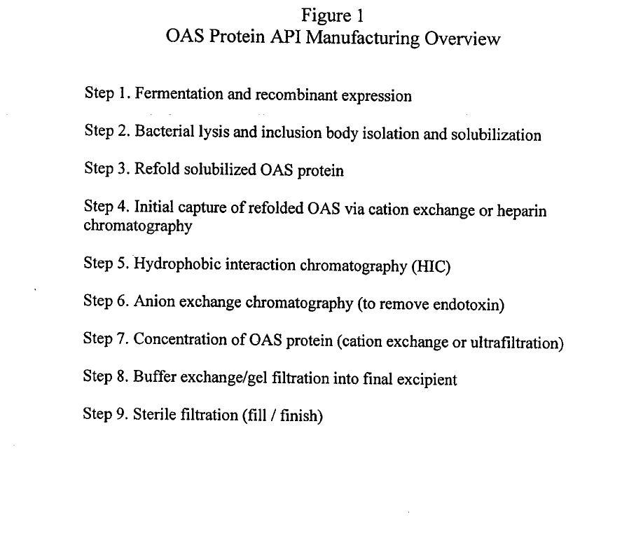

### DETAILED DESCRIPTION OF THE INVENTION

### Introduction and Definitions

We have demonstrated that mutations in the OAS genes confer resistance to virus infection. (U.S. Patent Application Ser. Nos. 60/513,888, filed Oct. 23, 2003; 60/542,373, filed Feb. 6, 2004; 60/554,758, filed Mar. 19, 2004; 60/560,524, filed Apr. 8, 2004; 60/578,323, filed Jun. 8, 2004; 60/605,243, filed Aug. 26, 2004; 10/972,135, filed Oct. 22, 2004; 60/677,680, filed May 4, 2005; and 60/710,704, filed Aug. 23, 2004, all of which are incorporated by reference herein.) Several novel forms of the OAS1, OAS2, and OAS3 genes have been cloned by us, and we have developed pharmaceutical compositions derived from these and other novel oligoadenylate synthetase protein forms (patent application Ser. No. 60/752,668 filed Dec. 21, 2005, and incorporated by reference herein). We have demonstrated that these pharmaceutical compositions are antiviral in vitro. We have further demonstrated that these pharmaceutical compositions promote cellular growth in certain cell lines. We have further demonstrated that these pharmaceutical compositions have a mitogenic effect. We have further demonstrated that these pharmaceutical compositions have the ability to enter a cell and remain enzymatically active in intracellular stores for several days or more. We have further demonstrated that these pharmaceutical compositions have broad antiviral activity. We have further demonstrated that these pharmaceutical compositions can be derivatized with polyethylene glycol and retain their enzymatic activity. We have demonstrated that the stability of the pharmaceutical compositions is dependent on the presence of reducing agents and stabilizing agents. We have demonstrated that bulk quantities of the pharmaceutical compositions can be manufactured using recombinant DNA technologies by expression in Escherichia coli. We have further demonstrated that these manufactured pharmaceutical compositions can be administered to mammals and produce no observable toxic effects. We have further demonstrated that these manufactured pharmaceutical compositions have good biodistribution and pharmacokinetic properties when administered to a mammal by injection.

The present invention describes methods for manufacturing oligoadenylate synthetase proteins for use as active pharmaceutical ingredients for the treatment of disease in mammals. The invention relates to the manufacture and use of the OAS proteins and polypeptides for the treatment of virus infection, inflammation, and neoplastic disease and to promote cellular growth and regeneration in mammals.

In reference to the detailed description, the following definitions are used:

A: adenine; C: cytosine; G: guanine; T: thymine (in DNA); and U: uracil (in RNA)

Allele: A variant of DNA sequence of a specific gene. In diploid cells a maximum of two alleles will be present, each in the same relative position or locus on homologous chromosomes of the chromosome set. When alleles at any one locus are identical the individual is said to be homozygous for that locus, and when they differ the individual is said to be heterozygous for that locus. Since different alleles of any one gene may vary by only a single base, the possible number of alleles for any one gene is very large. When alleles differ, one is often dominant to the other, which is said to be recessive. Dominance is a property of the phenotype and does not imply inactivation of the recessive allele by the dominant. In numerous examples the normally functioning (wild-type) allele is dominant to all mutant alleles of more or less defective function. In such cases the general explanation is that one functional allele out of two is sufficient to produce enough active gene product to support normal development of the organism (i.e., there is normally a two-fold safety margin in quantity of gene product).

Haplotype: One of many possible pluralities of Alleles, serially ordered by chromosomal localization and representing that set of Alleles carried by one particular homologous chromosome of the chromosome set.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Base Pair (bp): A partnership of adenine (A) with thymine (T), or of cytosine (C) with guanine (G) in a double stranded DNA molecule. In RNA, uracil (U) is substituted for thymine. When referring to RNA herein, the symbol T may be used interchangeably with U to represent uracil at a particular position in the RNA molecule.

Nucleic Acid: A polymer of nucleotides, either single or double stranded.

Polynucleotide: A polymer of single or double stranded nucleotides. As used herein “polynucleotide” and its grammatical equivalents will include the full range of nucleic acids. A polynucleotide will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention include primers, probes, RNA/DNA segments, oligonucleotides or “oligos” (relatively short polynucleotides), genes, vectors, plasmids, and the like.

Gene: A nucleic acid whose nucleotide sequence codes for an RNA or polypeptide. A gene can be either RNA or DNA.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Complementary Bases: Nucleotides that normally pair up when DNA or RNA adopts a double stranded configuration.

Complementary Nucleotide Sequence: A sequence of nucleotides in a single-stranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Hybridization: The pairing of substantially complementary nucleotide sequences (strands of nucleic acid) to form a duplex or heteroduplex by the establishment of hydrogen bonds between complementary base pairs. It is a specific, i.e. non-random, interaction between two complementary polynucleotides that can be competitively inhibited.

Nucleotide Analog: A purine or pyrimidine nucleotide that differs structurally from A, T, G, C, or U, but is sufficiently similar to substitute for the normal nucleotide in a nucleic acid molecule.

DNA Homolog: A nucleic acid having a preselected conserved nucleotide sequence and a sequence coding for a receptor capable of binding a preselected ligand.

Upstream: In the direction opposite to the direction of DNA transcription, and therefore going from 5′ to 3′ on the non-coding strand, or 3′ to 5′ on the mRNA.

Downstream: Further along a DNA sequence in the direction of sequence transcription or read out, that is traveling in a 3′- to 5′-direction along the non-coding strand of the DNA or 5′- to 3′-direction along the RNA transcript.

Stop Codon: Any of three codons that do not code for an amino acid, but instead cause termination of protein synthesis. They are UAG, UAA and UGA and are also referred to as a nonsense or termination codon.

Reading Frame: Particular sequence of contiguous nucleotide triplets (codons) employed in translation. The reading frame depends on the location of the translation initiation codon.

Intron: Also referred to as an intervening sequence, a noncoding sequence of DNA that is initially copied into RNA but is cut out of the final RNA transcript.

Protein or polypeptide: The term “protein” or “polypeptide” refers to a polymer of amino acids and does not refer to a specific length of the product. Peptides, oligopeptides, polypeptides, proteins, and polyproteins, as well as fragments of these, are included within this definition. The term may include post expression modifications of the protein, for example, glycosylations, acetylations, phosphorylations and the like. Included within the definition are, for example, proteins containing one or more analogs of an amino acid (including, for example, unnatural amino acids, etc.), proteins with substituted linkages, as well as other modifications known in the art, both naturally occurring and non-naturally occurring.

A “variant” is a polypeptide comprising a sequence which differs in one or more amino acid position(s) from that of a parent polypeptide sequence.

The term “parent polypeptide” is intended to indicate the polypeptide sequence to be modified in accordance with the present invention.

A “fragment” or “subsequence” is any portion of an entire sequence, up to but not including the entire sequence. Thus, a fragment or subsequence refers to a sequence of amino acids or nucleic acids that comprises a part of a longer sequence of amino acids (e.g., polypeptide) or nucleic acids (e.g., polynucleotide).

A polypeptide, nucleic acid, or other component is “isolated” when it is partially or completely separated from components with which it is normally associated (other peptides, polypeptides, proteins (including complexes, e.g., polymerases and ribosomes which may accompany a native sequence), nucleic acids, cells, synthetic reagents, cellular contaminants, cellular components, etc.), e.g., such as from other components with which it is normally associated in the cell from which it was originally derived. A polypeptide, nucleic acid, or other component is isolated when it is partially or completely recovered or separated from other components of its natural environment such that it is the predominant species present in a composition, mixture, or collection of components (i.e., on a molar basis it is more abundant than any other individual species in the composition). In some instances, the preparation consists of more than about 60%, 70% or 75%, typically more than about 80%, or preferably more than about 90% of the isolated species.

A “substantially pure” or “isolated” nucleic acid (e.g., RNA or DNA), polypeptide, protein, or composition also means where the object species (e.g., nucleic acid or polypeptide) comprises at least about 50, 60, or 70 percent by weight (on a molar basis) of all macromolecular species present. A substantially pure or isolated composition can also comprise at least about 80, 90, or 95 percent by weight of all macromolecular species present in the composition. An isolated object species can also be purified to essential homogeneity (contaminant species cannot be detected in the composition by conventional detection methods) wherein the composition consists essentially of derivatives of a single macromolecular species. The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or antisense.

A “recombinant polynucleotide” or a “recombinant polypeptide” is a non-naturally occurring polynucleotide or polypeptide which may include nucleic acid or amino acid sequences, respectively, from more than one source nucleic acid or polypeptide, which source nucleic acid or polypeptide can be a naturally occurring nucleic acid or polypeptide, or can itself have been subjected to mutagenesis or other type of modification. A nucleic acid or polypeptide may be deemed “recombinant” when it is synthetic or artificial or engineered, or derived from a synthetic or artificial or engineered polypeptide or nucleic acid. A recombinant nucleic acid (e.g., DNA or RNA) can be made by the combination (e.g., artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. A “recombinant polypeptide” often refers to a polypeptide that results from a cloned or recombinant nucleic acid. The source polynucleotides or polypeptides from which the different nucleic acid or amino acid sequences are derived are sometimes homologous (i.e., have, or encode a polypeptide that encodes, the same or a similar structure and/or function), and are often from different isolates, serotypes, strains, species, of organism or from different disease states, for example.

The term “recombinant” when used with reference, e.g., to a cell, polynucleotide, vector, protein, or polypeptide typically indicates that the cell, polynucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences that are not found in the native (non-recombinant) form of the cell or express native nucleic acid sequences that would otherwise be abnormally expressed, under-expressed, or not expressed at all. The term “recombinant” when used with reference to a cell indicates that the cell replicates a heterologous nucleic acid, or expresses a polypeptide encoded by a heterologous nucleic acid. Recombinant cells can contain coding sequences that are not found within the native (non-recombinant) form of the cell. Recombinant cells can also contain coding sequences found in the native form of the cell wherein the coding sequences are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, recombination, and related techniques.

The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated.

An “immunogen” refers to a substance capable of provoking an immune response, and includes, e.g., antigens, autoantigens that play a role in induction of autoimmune diseases, and tumor-associated antigens expressed on cancer cells. An immune response generally refers to the development of a cellular or antibody-mediated response to an agent, such as an antigen or fragment thereof or nucleic acid encoding such agent. In some instances, such a response comprises a production of at least one or a combination of CTLs, B cells, or various classes of T cells that are directed specifically to antigen-presenting cells expressing the antigen of interest.

An “antigen” refers to a substance that is capable of eliciting the formation of antibodies in a host or generating a specific population of lymphocytes reactive with that substance. Antigens are typically macromolecules (e.g., proteins and polysaccharides) that are foreign to the host.

An “adjuvant” refers to a substance that enhances an antigen's immune-stimulating properties or the pharmacological effect(s) of a drug. An adjuvant may non-specifically enhance the immune response to an antigen. “Freund's Complete Adjuvant,” for example, is an emulsion of oil and water containing an immunogen, an emulsifying agent and mycobacteria. Another example, “Freund's incomplete adjuvant,” is the same, but without mycobacteria.

A “vector” is a component or composition for facilitating cell transduction or transfection by a selected nucleic acid, or expression of the nucleic acid in the cell. Vectors include, e.g., plasmids, cosmids, viruses, YACs, bacteria, poly-lysine, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter. The nucleic acid to be transcribed is typically under the direction or control of the promoter.

The term “subject” as used herein includes, but is not limited to, an organism; a mammal, including, e.g., a human, non-human primate (e.g., baboon, orangutan, monkey), mouse, pig, cow, goat, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition suitable for pharmaceutical use in a subject, including an animal or human. A pharmaceutical composition generally comprises an effective amount of an active agent, “active pharmaceutical ingredient” and a carrier, including, e.g., a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

A “reducing agent” shall mean a compound that maintains sulfhydryl groups in the reduced state and reduces disulfide intra- or intermolecular bonds. Reducing agents include glutathione, dithioerythritol, dithiothreitol (DTT), or 2-mercaptoethanol.

“Dialysis” or “Ultrafiltration/Diafiltration” refers to standard methods for exchanging one buffer, e.g. the solubilization solution and/or the purification buffers, into a different buffer, e.g. the formulation solution to stabilize the solubilized protein and/or the final purified product. Ultrafiltration/Diafiltration may be used for both buffer exchange and to concentrate a protein.

“Inclusion” (or refractile) bodies shall mean dense, insoluble (i.e., not easily dissolved) protein aggregates (i.e., clumps) that are produced within the cells of certain microorganisms, generally by high expression levels of heterologous genes during fermentation. The term refractile bodies is used in some instances because their greater density (than the rest of the microorganism's body mass) causes light to be refracted (bent) when it is passed through them. This bending of light causes the appearance of very bright and dark areas around the refractile body and makes them visible under a microscope.

The term “refractile bodies” and “inclusion bodies” encompass insoluble cytoplasmic aggregates produced within a recombinant host organism wherein the aggregates contain, at least in part, a heterologous protein to be recovered.

“Disrupting” or “lysing” the host organism (cell) shall mean the process of breaking the bacterial cells to isolate the inclusion bodies or the recombinant polypeptides or proteins.

“Lysate” shall mean the residue from disruption of the host organism in the present method. A lysate arises, typically, from cytolysis, the dissolution of cells, particularly by destruction of their surface membranes. In some embodiments lysozymes lyse certain kinds of bacteria, by dissolving the polysaccharide components of the bacteria's cell wall. When that cell wall is weakened, the bacteria cell then bursts because osmotic pressure (inside that bacteria cell) is greater than the weakened cell wall can contain. In a particular embodiment, cells are lysed by digestion with Lysozyme or disrupted by three cycles of cell dispersion with a Teflon homogenizer followed by centrifugation. In another embodiment, cells are disrupted by several passes in a pressurized homogenizer (e.g., Gaulin) or a microfluidizer. Sonication is also used. Lysis can be performed on purified or unpurified cells in media.

“Chaotropic agent” refers to a compound that, in a suitable concentration in aqueous solution, is capable of changing the spatial configuration or conformation of proteins through alterations at the surface, rendering a protein to be isolated, soluble in the aqueous medium but without biological activity.

A “reducing agent” is the compound in an oxidation-reduction reaction that serves as the electron donor. Exemplary reducing agents include: 2-Mercaptoethylamine HCl, 2-mercaptoethanol, dithiothreitol, Ellman's reagent, Tris-(2-carboxyethyl)-phosphine hydrochloride, cysteine, and the like.

A “chelating agent” is a compound capable of forming coordinate bond with one or more metal ions.

“Stabilizing compounds” shall mean compounds such as sugars, surfactants such as polysorbate-10, polysorbate-80 and PEG, polyols, chelating agents, amino acids and polymers, which in combination will increase the solubility and biological activity of a protein. The structure of a protein is strongly influenced by pH. Thus, in the presence of solutions containing low quantities of OH.sup.− or H.sup.+ ions and stabilizers, ionization of the side chains occurs and solubilization takes place. Unfolding of tangled protein in inclusion bodies, at low concentration of the ions in the non-buffered aqueous solution, releases monomeric protein. Aqueous solutions containing osmolytic stabilizers such as sugars and polyols (polyhydric alcohols) provide protein stability, and thus the maintenance of solubility and biological activity of proteins. Such stability of protein structure by sugars is due to the preferential interaction of proteins with solvent components. The major effects of stabilizing compounds are on the viscosity and surface tension of the water. Many of these compounds include sugars, polyols, polysaccharides, neutral polymers, amino acids (glycine and alanine) and derivatives, and large dipolar molecules (i.e., trimethylamine N-oxide). Sugars such as mannitol and lactose maintain protein stability. Proteins are preferably hydrated in the presence of sugars. There is a positive change in the chemical potential of the protein induced by the addition of lactose and hence the stabilization of a protein. Polyols such as mannitol and glycerol are used also as protein stabilizers. Mannitol induces structure in the water molecules and stabilizes proteins by competing with water. This is believed due to the strong hydrophobic interaction between pairs of hydrophobic groups in the solutions of mannitol than in pure water. Without being bound by any specific theory, it is believed that Mannitol (and other polyols such as glycerol, sorbitol, arabitol and Xylitol) displace water allowing stabilization of hydrophobic interactions which are the major factor stabilizing the three-dimensional structure of proteins. Glycerol stabilizes proteins in solution, likely due to its ability to enter into and strengthen the water lattice structure. It is believed to prevent formation of precipitates by assisting preferential hydration and leads to the net stabilization of the native structure of proteins. Sorbitol likely competes for the hydration water of the protein, stabilizing the protein from denaturation, and amino acids such as L-arginine, taurine, sarcosine, glycine and serine, likely increase the surface tension of water, stabilizing proteins and suppressing aggregation.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoters include, for example, but are not limited to, IPTG-inducible promoters, bacteriophage T7 promoters and bacteriophage .lamda.p.sub.L. See Sambrook et al., Molecular Cloning: A Laboratory Manual, 3rd ed., Cold Spring Harbor Laboratory Press, Cold Spring Harbor, N.Y., 2001. A typical promoter will have three components, consisting of consensus sequences at −35 and −10 with a sequence of between 16 and 19 nucleotides between them (Lisset, S, and Margalit, H., Nucleic Acids Res. 21: 1512, 1993). Promoters of this sort include the lac, trp, trp-lac (tac) and trp-lac(trc) promoters. If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a eukaryotic regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner. Bacterial promoters have regulatory elements that bind and modulate the activity of the core promoter, such as operator sequences that bind activator or repressor molecules.

A “cloning vector” is a nucleic acid molecule, such as a plasmid, cosmid, or bacteriophage, which has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide resistance to antibiotic.

An “expression vector” is a nucleic acid molecule encoding a gene that is expressed in a host cell. Typically, an expression vector comprises a transcriptional promoter, a gene, an origin of replication, a selectable marker, and a transcriptional terminator. Gene expression is usually placed under the control of a promoter, and such a gene is said to be “operably linked to” the promoter. Similarly, a regulatory element and a core promoter are operably linked if the regulatory element modulates the activity of the core promoter. An expression vector may also be known as an expression construct.

The term “expression” refers to the biosynthesis of a gene product. For example, in the case of a structural gene, expression involves transcription of the structural gene into mRNA and the translation of mRNA into one or more polypeptides.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The terms “amino-terminal” or “N-terminal” and “carboxyl-terminal” or “C-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “fusion protein” is a hybrid protein expressed by a nucleic acid molecule comprising nucleotide sequences of at least two genes.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

The terms “OAS protein”, “OAS polypeptide”, and “polypeptide expressed by an OAS nucleotide” as utilized in the present invention with regard to producing active pharmaceutical ingredients shall mean any polypeptide having at least 40%, 45%, 50%, 55%, 60%, 65%, 70%, 75%, 80%, 85%, 90%, or 95% homology to known oligoadenylate synthetase polypeptides including but not limited to SEQ ID NO:9-16 regardless of whether said polypeptide has oligoadenylate synthetase activity.

The term “manufacture” or “manufacturing” as utilized in the present invention with regard to OAS proteins or polypeptides means the process of producing milligram or gram quantities of the desired proteins or polypeptides under conditions suitable for use as an active ingredient (i.e. active pharmaceutical ingredient) or active agent in a pharmaceutical composition.

Modes of Practicing the Invention

2′,5′-oligoadenylate synthetases (OAS) are a family of IFN-α-inducible, RNA dependent effector enzymes that synthesize short 2′ to 5′ linked oligoadenylate (2-5A) molecules from ATP. 2-5A molecules bind to and activate the RNAseL enzyme, which once activated, degrades viral and cellular RNAs and blocks cellular protein synthesis. OAS enzymes constitute an important part of the nonspecific immune defense against viral infections and have been used as a cellular marker for viral infection. In addition to the role in hepatitis C infection discussed herein, OAS activity is implicated in other disease states, particularly those in which a viral infection plays a role.

While specific pathogenic mechanisms are subjects of current analysis, viral infections are believed to play a role in the development of diseases such as diabetes. Lymphocytic OAS activity is significantly elevated in patients with type 1 diabetes, suggesting that OAS may be an important link between viral infections and disease development. In a study involving diabetic twins from monozygotic twin pairs, Bonnevie-Nielsen et al. (Clin Immunol. 2000 July; 96(1):11-8) showed that OAS is persistently activated in both recent-onset and long-standing type 1 diabetes. Continuously elevated OAS activity in type 1 diabetes is clearly different from a normal antiviral response and might indicate a chronic stimulation of the enzyme, a failure of down regulatory mechanisms, or an aberrant response to endogenous or exogenous viruses or their products.

A more direct link between a viral infection and the development of diabetes is exemplified by a number of studies showing that between 13 and 33% of patients with chronic hepatitis C have diabetes mellitus (type 2 diabetes), a level that is significantly increased compared with that in matched healthy controls or patients with chronic hepatitis B (Knobler et al. Am J Gastroenterol. 2003 December; 98(12):2751-6). While OAS has not to date been reported to play a role in the development of diabetes mellitus following hepatitis C infection, it may be a useful marker for the antiviral response system. A further published study has shown that OAS plays an essential role in wound healing and its pathological disorders, particularly in the case of venous ulcers and diabetes-associated poorly-healing wounds (WO 02/090552). In the case of poor wound healing, OAS mRNA levels in the affected tissues were reduced, rather than elevated as in lymphocytes derived from patients suffering from type 1 diabetes. These findings point to OAS as an etiologically important marker of immune reactions in diabetes and diabetes-related wound healing.

OAS may also play an intermediary role in cell processes involved in prostate cancer. A primary biochemical function of OAS is to promote the activity of RNaseL, a uniquely-regulated endoribonuclease that is enzymatically stimulated by 2-5A molecules. RNaseL has a well-established role in mediating the antiviral effects of IFN, and is a strong candidate for the hereditary prostate cancer 1 allele (HPC1). Mutations in RNaseL have been shown to predispose men to an increased incidence of prostate cancer, which in some cases reflect more aggressive disease and/or decreased age of onset compared with non RNaseL-linked cases. Xiang et al. (Cancer Res. 2003 Oct. 15; 63(20):6795-801) demonstrated that biostable phosphorothiolate analogs of 2-5A induced RNaseL activity and caused apoptosis in cultures of late-stage metastatic human prostate cancer cell lines. Their findings suggest that the elevation of OAS activity with a concurrent increase in 2-5A levels may facilitate the destruction of cancer cells through a potent apoptotic pathway.

OAS may further play a role in normal cell growth regulation, either through its regulation of RNaseL or through another as yet undiscovered pathway. There is considerable evidence to support the importance of OAS in negatively regulating cell growth. Rysiecki et al. (J. Interferon Res. 1989 December; 9(6):649-57) demonstrated that stable transfection of human OAS into a glioblastoma cell line results in reduced cellular proliferation. OAS levels have also been shown to be measurable in several studies comparing quiescent versus proliferating cell lines (e.g. Hassel and Ts'O, Mol. Carcinog. 1992; 5(1):41-51 and Kimchi et al., Eur J. Biochem. 1981; 114(1):5-10) and in each case the OAS levels were greatest in quiescent cells. Other studies have shown a correlation between OAS level and cell cycle phase, with OAS levels rising sharply during late S phase and then dropping abruptly in G2 (Wells and Mallucci, Exp Cell Res. 1985 July; 159(1):27-36). Several studies have shown a correlation between the induction of OAS and the onset of antiproliferative effects following stimulation with various forms of interferon (see Player and Torrence, Pharmacol Ther. 1998 May; 78(2):55-113). Induction of OAS has also been shown during cell differentiation (e.g. Salzberg et al., J Cell Sci. 1996 June; 109(Pt 6):1517-26 and Schwartz and Nilson, Mol Cell Biol. 1989 September; 9(9):3897-903). Other reports of induction of OAS by platelet derived growth factor (PDGF) (Zullo et al. Cell. 1985 December; 43(3 Pt 2):793-800) and under conditions of heat-shock induced growth (Chousterman et al., J Biol. Chem. 1987 Apr. 5; 262(10):4806-11) lead to the hypothesis that induction of OAS is a normal cell growth control mechanism.

Previous efforts to purify OAS proteins are quite different from the process described in the current specification and fail to produce substantially pure, active pharmaceutical ingredients for pharmaceutical compositions. Chebath and colleagues (Mory et al (1989) J. Interferon Res. 9:295-304) used ion exchange and affinity chromatography to purify OAS proteins produced by recombinant expression in E. coli. However, the resulting proteins were of limited purity, depended on a costly affinity purification step, and did not result in OAS protein products appropriate for pharmaceutical compositions; nor did the process operate on a scale appropriate for commercial manufacture. Sarkar and Sen were unable to produce enzymatically active OAS proteins in E. coli and therefore used a baculovirus expression system in insect cells (S, N. Sarkar and G. C. Sen (1998) Methods 15:233-242). The authors isolated soluble OAS protein via lysis of the insect cells, and purified OAS proteins using size fractionation (Sephacryl S-300) and phosphocellulose affinity purification columns. Again, the process developed was not of appropriate scale, cost-effectiveness or purity to produce active ingredients for pharmaceutical compositions in a commercial setting.

We have previously demonstrated that a variety of oligoadenylate synthetase proteins can be used as antiviral agents. The present invention fulfils a need in the art by providing methods for the manufacture of OAS proteins for use as active ingredients in pharmaceutical compositions for the treatment of disease.

Recombinant Expression and Purification of OAS Proteins

Recombinant methods for producing and isolating OAS polypeptides or proteins are described herein. One such method comprises introducing into a population of cells any nucleic acid, which is operatively linked to a regulatory sequence effective to produce the encoded OAS polypeptide, culturing the cells in a culture medium to express the polypeptide, and isolating the polypeptide from the cells or from the culture medium. An amount of OAS encoding nucleic acid sufficient to facilitate uptake by the cells (transfection) and/or expression of the OAS polypeptide is utilized. The nucleic acid is introduced into such cells by any delivery method as is known in the art, including, e.g., injection, gene gun, passive uptake, etc. As one skilled in the art will recognize, the nucleic acid may be part of a vector, such as a recombinant expression vector, including a DNA plasmid vector, or any vector as known in the art. The nucleic acid or vector comprising a nucleic acid encoding an OAS polypeptide may be prepared and formulated by standard recombinant DNA technologies and isolation methods as known in the art. Such a nucleic acid or expression vector may be introduced into a population of cells of a mammal in vivo, or selected cells of the mammal (e.g., tumor cells) may be removed from the mammal and the nucleic acid expression vector introduced ex vivo into the population of such cells in an amount sufficient such that uptake and expression of the encoded polypeptide results. Or, a nucleic acid or vector comprising a nucleic acid encoding an OAS polypeptide is produced using cultured cells in vitro. In one aspect, the method of producing an OAS polypeptide comprises introducing into a population of cells a recombinant expression vector comprising any nucleic acid encoding an OAS polypeptide in an amount and formula such that uptake of the vector and expression of the encoded polypeptide will result; administering the expression vector into a mammal by any introduction/delivery format described herein; and isolating the polypeptide from the mammal or from a byproduct of the mammal.

The invention provides isolated or recombinant nucleic acids (also referred to herein as polynucleotides), collectively referred to as “nucleic acids (or polynucleotides) of the invention”, which encode OAS polypeptides. The polynucleotides of the invention are useful in a variety of applications. As discussed above, the polynucleotides are useful in producing OAS polypeptides.

Any of the polynucleotides of the invention (which includes those described above) may encode a fusion protein comprising at least one additional amino acid sequence, such as, for example, a secretion/localization sequence, a sequence useful for solubilization or immobilization (e.g., for cell surface display) of the OAS polypeptide, a sequence useful for detection and/or purification of the OAS polypeptide (e.g., a polypeptide purification subsequence, such as an epitope tag, a polyhistidine sequence, and the like), or a sequence for increasing cellular uptake. In another aspect, the invention provides cells comprising one or more of the polynucleotides of the invention. Such cells may express one or more OAS polypeptides encoded by the polynucleotides of the invention.

The invention also provides vectors comprising any of the polynucleotides of the invention. Such vectors may comprise a plasmid, a cosmid, a phage, a virus, or a fragment of a virus. Such vectors may comprise an expression vector, and, if desired, the nucleic acid is operably linked to a promoter, including those discussed herein and below.

The present invention also includes recombinant constructs comprising one or more of the nucleic acid sequences as broadly described above. The constructs comprise a vector, such as, a plasmid, a cosmid, a phage, a virus, a bacterial artificial chromosome (BAC), a yeast artificial chromosome (YAC), and the like, into which a nucleic acid sequence of the invention has been inserted, in a forward or reverse orientation. In some instances, the construct further comprises regulatory sequences, including, for example, a promoter, operably linked to the nucleic acid sequence. Large numbers of suitable vectors and promoters are known to those of skill in the art, and are commercially available.

General texts that describe molecular biological techniques useful herein, including the use of vectors, promoters and many other relevant topics, include Berger, supra; Sambrook (1989), supra, and Ausubel, supra. Examples of techniques sufficient to direct persons of skill through in vitro amplification methods, including the polymerase chain reaction (PCR) the ligase chain reaction (LCR), Q beta-replicase amplification and other RNA polymerase mediated techniques (e.g., NASBA), e.g., for the production of the homologous nucleic acids of the invention are found in Berger, Sambrook, and Ausubel, all supra, as well as Mullis et al. (1987) U.S. Pat. No. 4,683,202; PCR Protocols: A Guide to Methods and Applications (Innis et al., eds.) Academic Press Inc. San Diego, Calif. (1990) (“Innis”); Arnheim & Levinson (Oct. 1, 1990) C&EN 36-47; The Journal Of NIH Research (1991) 3:81-94; (Kwoh et al. (1989) Proc Natl Acad Sci USA 86:1173-1177; Guatelli et al. (1990) Proc Natl Acad Sci USA 87:1874-1878; Lomeli et al. (1989) J Clin Chem 35:1826-1831; Landegren et al. (1988) Science 241:1077-1080; Van Brunt (1990) Biotechnology 8:291-294; Wu and Wallace (1989) Gene 4:560-569; Barringer et al. (1990) Gene 89:117-122, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kilobases (kb) are generated. One of skill will appreciate that essentially any RNA can be converted into a double stranded DNA suitable for restriction digestion, PCR expansion and sequencing using reverse transcriptase and a polymerase. See Ausubel, Sambrook and Berger, all supra.

The present invention also provides host cells that are transduced with vectors of the invention, and the production of OAS polypeptides of the invention by recombinant techniques. Host cells are genetically engineered (e.g., transduced, transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying genes. The culture conditions, such as temperature, pH, and the like, are those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein.

OAS polypeptides can also be produced in non-animal cells such as plants, yeast, fungi, bacteria and the like. In addition to Sambrook, Berger and Ausubel, details regarding cell culture are found in, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York); Atlas & Parks (eds.) The Handbook of Microbiological Media (1993) CRC Press, Boca Raton, Fla.

The polynucleotides of the present invention and fragments thereof may be included in any one of a variety of expression vectors for expressing an OAS polypeptide. Such vectors include chromosomal, nonchromosomal and synthetic DNA sequences, e.g., derivatives of SV40, bacterial plasmids, phage DNA, baculovirus, yeast plasmids, vectors derived from combinations of plasmids and phage DNA. Any vector that transduces genetic material into a cell, and, if replication is desired, which is replicable and viable in the relevant host can be used.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda PL promoter, CMV promoter, and other promoters known to control expression of genes in prokaryotic or eukaryotic cells. The expression vector also contains a ribosome binding site for translation initiation, and a transcription terminator. The vector optionally includes appropriate sequences for amplifying expression, e.g., an enhancer. In addition, the expression vectors optionally comprise one or more selectable marker genes to provide a phenotypic trait for selection of transformed host cells, such as dihydrofolate reductase or neomycin resistance for eukaryotic cell culture, or such as tetracycline, kanamycin or ampicillin resistance in E. coli.

The vector containing the appropriate DNA sequence encoding an OAS polypeptide of the invention, as well as an appropriate promoter or control sequence, may be employed to transform an appropriate host to permit the host to express the polypeptide. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 or Bowes melanoma; plant cells, etc. It is understood that not all cells or cell lines need to be capable of producing fully functional OAS polypeptides or fragments thereof; for example, antigenic fragments of the polypeptide may be produced in a bacterial or other expression system. The invention is not limited by the host cells employed.

In bacterial systems, a number of expression vectors may be selected depending upon the use intended for the OAS polypeptide or fragment thereof. For example, when large quantities of a polypeptide or fragments thereof are needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the nucleotide coding sequence may be ligated into the vector in-frame with sequences for the amino-terminal Met and the subsequent 7 residues of beta-galactosidase so that a hybrid protein is produced; pIN vectors (Van Heeke & Schuster (1989) J Biol Chem 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH may be used for production of the polypeptides of the invention. For reviews, see Ausubel, supra, Berger, supra, and Grant et al. (1987) Methods in Enzymology 153:516-544.

In mammalian host cells, a number of expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome results in a viable virus capable of expressing an OAS polypeptide in infected host cells (Logan and Shenk (1984) Proc Natl Acad Sci USA 81:3655-3659). In addition, transcription enhancers, such as the rous sarcoma virus (RSV) enhancer, are used to increase expression in mammalian host cells. Host cells, media, expression systems, and methods of production include those known for cloning and expression of various mammalian proteins.

Specific initiation signals can aid in efficient translation of a polynucleotide coding sequence of the invention and/or fragments thereof. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous nucleic acid transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (see, e.g., Scharf D. et al. (1994) Results Probl Cell Differ 20:125-62; and Bittner et al. (1987) Methods in Enzymol 153:516-544).

Polynucleotides encoding OAS polypeptides can also be fused, for example, in-frame to nucleic acids encoding a secretion/localization sequence, to target polypeptide expression to a desired cellular compartment, membrane, or organelle, or to direct polypeptide secretion to the periplasmic space or into the cell culture media. Such sequences are known to those of skill, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

In a further aspect, the present invention relates to host cells containing any of the above-described nucleic acids, vectors, or other constructs of the invention. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-dextran mediated transfection, electroporation, gene or vaccine gun, injection, or other common techniques (see, e.g., Davis, L., Dibner, M., and Battey, I. (1986) Basic Methods in Molecular Biology) for in vivo, ex vivo or in vitro methods.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre” or a “prepro” form of the protein may also be important for correct insertion, folding and/or function. Different host cells such as E. coli, Bacillus sp., yeast or mammalian cells such as CHO, HeLa, BHK, MDCK, HEK 293, W138, etc. have specific cellular machinery and characteristic mechanisms for such post-translational activities and may be chosen to ensure the correct modification and processing of the introduced foreign protein.

Stable expression can be used for long-term, high-yield production of recombinant OAS proteins. For example, cell lines which stably express a polypeptide of the invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

Host cells transformed with a nucleotide sequence encoding an OAS polypeptide are optionally cultured under conditions suitable for the expression and recovery of the encoded protein from cell culture. The polypeptide produced by a recombinant cell may be secreted, membrane-bound, or contained intracellularly, depending on the sequence and/or the vector used. As will be understood by those of skill in the art, expression vectors containing polynucleotides encoding polypeptides of the invention can be designed with signal sequences which direct secretion of the mature polypeptides through a prokaryotic or eukaryotic cell membrane.

The polynucleotides of the present invention optionally comprise a coding sequence fused in-frame to a marker sequence which, e.g., facilitates purification and/or detection of the encoded polypeptide. Such purification subsequences include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I. et al. (1984) Cell 37:767), maltose binding protein sequences, the FLAG epitope utilized in the FLAGS extension/affinity purification system, and the like. The inclusion of a protease-cleavable polypeptide linker sequence between the purification domain and the polypeptide sequence is useful to facilitate purification.

For example, one expression vector possible to use in the compositions and methods described herein provides for expression of a fusion protein comprising an OAS polypeptide fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281) while the enterokinase cleavage site provides a method for separating the desired polypeptide from the polyhistidine region. pGEX vectors (Promega; Madison, Wis.) are optionally used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Eukaryotic or microbial cells employed in expression of the proteins can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian) and archebacterial origin. See, e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, New York; Humason (1979) Animal Tissue Techniques, fourth edition W.H. Freeman and Company; and Ricciardelli et al. (1989) In vitro Cell Dev Biol 25:1016-1024. For plant cell culture and regeneration see, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy (ed.) Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue from Sigma-Aldrich, Inc (St Louis, Mo.) (“Sigma-LSRCCC”) and, e.g., the Plant Culture Catalogue and supplement also from Sigma-Aldrich, Inc (St Louis, Mo.) (“Sigma-PCCS”).

OAS polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature OAS protein or fragments thereof. Finally, high performance liquid chromatography (HPLC) can be employed in the final purification steps. In addition to the references noted, supra, a variety of purification methods are well known in the art, including, e.g., those set forth in Sandana (1997) Bioseparation of Proteins, Academic Press, Inc.; Bollag et al. (1996) Protein Methods, 2.sup.nd Edition Wiley-Liss, New York; Walker (1996) The Protein Protocols Handbook Humana Press, New Jersey; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3.sup.rd Edition Springer Verlag, New York; Janson and Ryden (1998) Protein Purification: Principles, High Resolution Methods and Applications, Second Edition Wiley-VCH, New York; and Walker (1998) Protein Protocols on CD-ROM Humana Press, New Jersey.

### Embodiments

OAS Protein Active Pharmaceutical Ingredient (API) Expression and Fermentation

In an exemplary embodiment, an E. coli strain containing a lysogen of λDE3, and therefore carrying a chromosomal copy of the T7 RNA polymerase gene under the control of the lacUV5 promoter, is transformed with a bacterial expression vector containing an isopropyl beta-D-1-thiogalactopyranoside (IPTG)-inducible promoter encoding a nucleic acid sequence corresponding to one or more OAS proteins or polypeptides. Cultures are grown in Luria broth medium supplemented with 15 μg/mL kanamycin at 37° C. When the OD600 reaches >0.6, the temperature is reduced to 18° C. and the cells are induced with 0.5 mM IPTG for 17 hours. The above low temperature induction favors the expression of primarily full-length, soluble OAS proteins outside of inclusion bodies. The bacterial cells are then resuspended in buffer containing 50 mM NaH₂PO₄, pH 8, 300 mM NaCl, 20 mM imidazole, 10% glycerol, 0.1% NP40, 2 mM DTT and protease inhibitors, lysed in a Gaulin homogenizer, and centrifuged to remove cell debris before protein purification.

In another exemplary embodiment, OAS proteins are expressed by cloning into the pET9d expression vector and transformed into the BL21(DE3) host E. coli strain. Recombinant bacterial cultures are grown in Luria broth to an OD(600 nm) of about 0.6 and induced to express OAS proteins by the addition of IPTG to a final concentration of 1 mM for 3-4 hours at 37° C. Under these induction conditions, a majority of full length OAS proteins are found in an insoluble form in inclusion bodies. Bacterial cell cultures are centrifuged to collect the cell pellet at 9000×g. Cell pellets are resuspended in 50 mM NaH₂PO₄, 0.5% Triton X-100, 100 mM NaCl, 1 mM EDTA, pH 7.4. Lysozyme is added to 1 mg/mL and sonication is used to disrupt the cell membrane. DNAse and RNAse are added to a final concentration of 50 ug/mL each to reduce the viscosity of the cell lysate. An equal volume of a solution of 50 mM NaH₂PO₄, 5% Triton X-100, 2 M urea, 100 mM NaCl, 1 mM EDTA, pH 7.4 is added and the mixture is stirred for 30 minutes at room temperature. The lysate is snap-frozen, thawed, and centrifuged at 9000×g to recover inclusion bodies. Inclusion bodies are washed one time in a solution of 50 mM NaH₂PO₄, 5% Triton X-100, 2 M urea, 100 mM NaCl, 1 mM EDTA, pH 7.4 followed by centrifugation at 9000×g for 30 minutes. Additional inclusion body washes are performed using phosphate buffered saline (PBS) pH 7.4 followed by centrifugation as above. Inclusion body pellets are solubilized by the addition of 50 mL of a solution of 50 mM NaH₂PO₄, 6 M guanidine HCl, pH 8.0 for every 2.5 grams of wet inclusion body pellet. Dithiothreitol (DTT) is added to a final concentration of 50 mM. The mixture is stirred at room temperature for at least two hours or until clear. Sonication is used to improve the clarity and solubilization of inclusion bodies. Bacterial expression of OAS proteins can be evaluated by SDS-PAGE of solubilized inclusion body preparations. Approximately 50% or more of solubilized inclusion body protein is found to be OAS protein.

In one embodiment, the bacterial strain used is a derivative of BL21. In another embodiment, bacteria are grown in terrific broth or a synthetic media. In a still further embodiment, media are supplemented with buffers, amino acids, sugars, or other carbon sources. In a still further embodiment, bacteria are grown in shaker flasks, seed cultures, or fermenters. Bacterial cultures are grown to a variety of cell densities before induction of OAS protein expression, depending on culture conditions. Cell density at the time of induction—as measured by optical density at a wavelength of 600 nm—is for example about 0.2, 0.3, 0.4, 0.5, 0.6, 0.7, 0.8, 0.9, 1.0, 1.2, 1.5, 2.0, or 2.5, for example 5.0 or 10.0. Bacteria are grown under a variety of selective conditions, depending on the recombinant protein expression vector used and the host E. coli strain. In preferred embodiments, bacteria are grown in the presence of about, for example, 1 ug/mL, 5 ug/mL, 10 ug/mL, 15 ug/mL, 20 ug/mL, 50 ug/mL, or 100 ug/mL kanamycin. In a still further embodiment, bacterial cultures can be grown at any temperature between 30° C. and 40° C.

Induction is performed under a variety of concentrations of the IPTG inducer, such as for example, about 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.5 mM, 2.0 mM, 2.5 mM, 3.0 mM, 4.0 mM, or 5.0 mM. In a still further embodiment, OAS protein induction can be performed at temperatures between 4° C. and 40° C. and at times between 30 minutes and 48 hours. In a still further embodiment, bacterial cultures at appropriate densities are induced for OAS protein expression for 3-4 hours at 37° C. with a final concentration of 1 mM IPTG or at 10 hours at 37° C. with a final concentration of 1 mM IPTG. A variety of induction temperatures and times are appropriate for OAS protein expression. Shorter induction times and higher temperatures favor the expression of full-length insoluble OAS proteins into inclusion bodies. Longer induction periods and lower temperatures favor expression of soluble OAS protein outside of inclusions bodies. Induction of OAS proteins is carried out in a fermenter or under fermentation conditions where end of induction OD600 values reach as high as 50, for example, 60, 70, 80, 90, about 100, 110, 120, 130, 140, or 150 units. At the end of induction, cells are collected by a variety of methods including centrifugation and filtration, or lysed or homogenized directly in media without separation. As one skilled in the art will recognize, a variety of cell collection methods are envisioned by the specification. OAS proteins exceed 10% of total cellular protein, for example 11%, 12%, 13%, about 15%, about 20%.

Bacterial cells and inclusion bodies containing recombinant OAS proteins are collected, washed, lysed, and solubilized under a variety of buffer and solution conditions. A variety of buffers over a range of pKₐ values are used for buffering solutions including N-(2-acetamido)-2-aminoethane sulfonic acid (ACES), imidazole, phosphate, N-morpholinopropane sulfonic acid (MOPS)N-tris(hydroxymethyl)methyl-2-aminoethane sulfonic acid (TES), triethanolamine, Tris(hydroxymethyl)aminomethane (TRIS), N-Tris(hydroxymethyl)methyl-glycine (Tricine), Tris(hydroxymethyl)aminopropoane (TAPS), N-(2-Hydroxyethyl)piperazine-N′-(2-ethanesulfonic acid) (HEPES),2-amino-2-methyl-1,3-propanediol, diethanolamine, boric acid, NaH₂PO₄, and ethanolamine Buffers are used at a variety of concentrations, such as for example, 1 mM, 5 mM, 10 mM, about 25 mM, about 50 mM, about 100 mM, about 200 mM and at a variety of pH values, such as for example, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, and sodium thiocyanate, at concentrations of about 10 mM, about 25 mM, about 50 mM, about 75 mM, about 100 mM, about 200 mM, about 300 mM, about 500 mM, about 700 mM, about 1M. Chaotropic agents are used to enhance washing and solubilization of OAS protein containing inclusion bodies, such chaotropic agents include: urea, guanidine HCl, thiourea, and the like, at concentrations such as for example about 0.25M, 0.5M, 1.0M, 2.0M, 3.0M, 4.0M, 5.0M, 6.0M, 7.0M, such as for example 8.0M and above including near saturation solutions. A variety of detergents are added to facilitate bacterial cell lysis and inclusion body washing and solubilization, such as Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), aminosulfobetaine-16 (ASB-16), 3-(1-pyridinio)-1-propanesulfonate (NDSB 201), and dodecyl sulfate, at concentrations of for example, 0.1% w/v, 0.2% w/v, 0.3% w/v, 0.4% w/v, 0.5% w/v, about 1% w/v, about 5% w/v, about 10% w/v, more than 10% w/v. In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Stabilizing agents including surfactants, sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added during cell lysis and inclusion body solubilization to stabilize OAS proteins. Thiol-protective or reducing agents are added to prevent errant disulfide bond formation, such thiol-protective groups including dithiothreitol (DTT), dithioerythritol (DTE),2-mercaptoethanol, 2-3-dimercaptopropanol, tributylphosphine (TBP), tris-carboxyethylphosphine (TCEP), thioglycolate, glutathione, and cysteine at concentrations of between 0.5 and 100 mM, such as for example, 1 mM, about 5 mM, about 10 mM, about 25 mM, about 50 mM, about 100 mM.

Enzymes are added to aid in cell lysis, such as for example lysozyme at concentrations of 1 mg/mL, about 2 mg/mL, about 5 mg/mL, or about 10 mg/mL. Mechanical disruption is used to lyse bacterial cells and to clarify and solubilize inclusion bodies, such appropriate mechanical methods include: sonication, Gaulin homogenization, use of blenders, use of French pressure cells, Dounce homogenization, polytron homogenization, Potter-Elvehjem homogenization, and freeze/thaw methods as those skilled in the art will recognize. Heat, different pH buffers, different reductants and high pressure are also used to enhance solubilization of inclusion bodies. Numerous techniques are used to separate inclusion bodies from other cellular proteins and debris including: centrifugation, membrane filtration, tangential flow filtration, hollow fiber filtration, and expanded bed absorption. Inclusion bodies can also be washed in water.

Exemplary inclusion body solubilization solutions include: 6M guanidine hydrochloride, 50 mM sodium phosphate, 100 mM DTT, 10 mM EDTA, pH8.0; 8M guanidine hydrochloride, 50 mM sodium phosphate, 100 mM DTT, 10 mM EDTA, pH8.0; 8.4M urea, 2M guanidine hydrochloride, 50 mM sodium phosphate, 100 mM DTT, 10 mM EDTA, pH8.0; 8M urea, 4M guanidine hydrochloride, 50 mM sodium phosphate, 100 mM DTT, 10 mM EDTA, pH8.0; and 6M urea, 4M guanidine hydrochloride, 50 mM sodium phosphate, 100 mM DTT, 10 mM EDTA, pH8.0.

OAS Protein API Refolding of Insoluble Preparations

In an exemplary embodiment, solubilized inclusion bodies are adjusted to a final protein concentration of 10 to 15 mg/mL prior to pulse dilution into an appropriate refolding buffer. Solubilized inclusion bodies with final protein concentrations greater than 30 mg/mL demonstrate poor refolding potential. OAS protein refolding is performed by pulse dilution at 4° C. and at a flow-rate of 0.2 mL/minute over a 16 hour period into a stirred solution composed of 50 mM NaH₂PO₄, 300 mM guanidine HCl, 0.5% Tween-20®, 10% glycerol, 5 mM β-mercaptoethanol, pH 8.0. Both the solubilized inclusion bodies and the refolding solution are precooled to 4° C. The final total dilution of solubilized inclusion bodies into refolding solution is approximately 1:20. In exemplary embodiments, detergent is used to facilitate proper refolding of the OAS protein API. CHAPS at 0.1%, 0.5% and 1% w/v is used, as well as Tween-20® at a final concentration of between 0.1% and 1.0% w/v. 1% Tween-20® is shown to reduce aggregation of the OAS protein API during refolding. Refolding solutions at pH 8.0 perform better than refolding solutions at pH 6.8. Likewise, refolding solutions containing 2-mercaptoethanol as a reducing agent perform better than refolding solutions containing DTT. Refolding performed at 4° C. is more efficient than a refolding process performed at room temperature. The presence of chaotropic agents and high salt also enhance OAS protein refolding; for example the addition of 300 mM NaCl and 300 mM guanidine HCl enhances protein refolding efficiency. In an exemplary embodiment, fold-dilutions of solubilized inclusion bodies between 10 and 120 produce large amounts of properly folded and highly active OAS protein API. Refolding efficiencies of greater than 40% are achieved. The temperature of refolding can be varied between 4° C. and 16° C., for example 5° C., 6° C., 7° C., 8° C., about 10° C., 11° C., 12° C., about 14° C., about 16° C. Pulse dilution can occur over 1 to 24 hours, for example around 1, 2, 4, 6, 8, 10, about 12, about 16, around 20, around 24 hours or more. Exemplary refolding buffers include but are not limited to: 300 mM GuHCl, 50 mM NaH₂PO₄, 2 mM DTT, 1 mM EDTA, 10% glycerol, 0.5% Tween 20, pH 8.0; 400 mM GuHCl, 50 mM NaH₂PO₄, 2 mM DTT, 1 mM EDTA, 10% glycerol, 0.5% Tween 20, pH 8.0; 500 mM L-Arginine, 50 mM NaH₂PO₄, 2 mM DTT, 1 mM EDTA, 10% glycerol, 0.5% Tween 20, pH 8.0; 2 M Urea, 50 mM NaH₂PO₄, 2 mM DTT, 1 mM EDTA, 10% glycerol, 0.5% Tween 20, pH 8.0; 500 mM L-Histidine, 50 mM NaH₂PO₄, 2 mM DTT, 1 mM EDTA, 10% glycerol, 0.5% Tween 20, pH 8.0; 300 mM L-Histidine, 50 mM NaH₂PO₄, 2 mM DTT, 1 mM EDTA, 10% glycerol, 0.5% Tween 20, pH 8.0; and 500 mM NaCl, 50 mM NaH₂PO₄, 2 mM DTT, 1 mM EDTA, 10% glycerol, 0.5% Tween 20, pH 8.0.

Further exemplary refold buffers include but are not limited to: 100 mM L-Arginine, 300 mM L-Histidine, 50 mM NaH₂PO₄, 2 mM DTT, 1 mM EDTA, 10% glycerol, 0.5% Tween 20, pH 8.0; 200 mM L-Arginine, 300 mM L-Histidine, 50 mM NaH₂PO₄, 2 mM DTT, 1 mM EDTA, 10% glycerol, 0.5% Tween 20, pH 8.0; 300 mM L-Arginine, 300 mM L-Histidine, 50 mM NaH₂PO₄, 2 mM DTT, 1 mM EDTA, 10% glycerol, 0.5% Tween 20, pH 8.0; 400 mM L-Arginine, 300 mM L-Histidine, 50 mM NaH₂PO₄, 2 mM DTT, 1 mM EDTA, 10% glycerol, 0.5% Tween 20, pH 8.0; 500 mM L-Arginine, 300 mM L-Histidine, 50 mM NaH₂PO₄, 2 mM DTT, 1 mM EDTA, 10% glycerol, 0.5% Tween 20, pH 8.0; 500 mM L-Arginine, 500 mM NaCl, 50 mM NaH₂PO₄, 2 mM DTT, 1 mM EDTA, 10% glycerol, 0.5% Tween 20, pH 8.0; 300 mM L-Histidine, 500 mM NaCl, 50 mM NaH₂PO₄, 2 mM DTT, 1 mM EDTA, 10% glycerol, 0.5% Tween 20, pH 8.0; 800 mM L-Arginine, 300 mM L-Histidine, 50 mM NaH₂PO₄, 2 mM DTT, 1 mM EDTA, 10% glycerol, 0.5% Tween 20, pH 8.0; 500 mM L-Arginine, 300 mM L-Histidine, 500 mM Urea, 50 mM NaH₂PO₄, 2 mM DTT, 1 mM EDTA, 10% glycerol, 0.5% Tween 20, pH 8.0; 500 mM L-Arginine, 300 mM L-Histidine, 200 mM NaCl, 50 mM NaH₂PO₄, 2 mM DTT, 1 mM EDTA, 10% glycerol, 0.5% Tween 20, pH 8.0; and 300 mM L-Arginine, 200 mM L-Histidine, 50 mM NaH₂PO₄, 2 mM DTT, 1 mM EDTA, 10% glycerol, 0.5% Tween 20, pH 8.0.

Further exemplary refold buffers include but are not limited to: 300 mM L-Arginine, 200 mM L-Histidine, 10% sucrose, 50 mM NaH₂PO₄, 2 mM DTT, 1 mM EDTA, 0.5% Tween 20, pH 8.0; 300 mM L-Arginine, 200 mM L-Histidine, 20% sucrose, 50 mM NaH₂PO₄, 2 mM DTT, 1 mM EDTA, 0.5% Tween 20, pH 8.0; 300 mM L-Arginine, 200 mM L-Histidine, 20% glycerol, 50 mM NaH₂PO₄, 2 mM DTT, 1 mM EDTA, 0.5% Tween 20, pH 8.0; 100 mM L-Arginine, 100 mM L-Histidine, 2% Tween 20, 50 mM NaH₂PO₄, 2 mM DTT, 1 mM EDTA, 10% glycerol, pH 8.0; 100 mM L-Arginine, 100 mM L-Histidine, 20% glycerol, 2% Tween 20, 50 mM NaH₂PO₄, 2 mM DTT, 1 mM EDTA, pH 8.0; 100 mM L-Arginine, 100 mM L-Histidine, 20% glycerol, 2% Tween 20, 10 mM DTT, 50 mM NaH₂PO₄, 1 mM EDTA, pH 8.0; 100 mM L-Arginine, 100 mM L-Histidine, 30% glycerol, 2% Tween 20, 10 mM DTT, 50 mM NaH₂PO₄, 1 mM EDTA, pH 8.0; and 100 mM L-Arginine, 100 mM L-Histidine, 30% sucrose, 2% Tween 20, 10 mM DTT, 50 mM NaH₂PO₄, 1 mM EDTA, pH 8.0.

In another embodiment, immediate dilution is used for refolding of insoluble OAS proteins. In a still further embodiment, buffer exchange through dialysis, tangential flow filtration and gel filtration are used to mediate OAS protein refolding.

In another embodiment alternative buffers over a range of pKₐ values are used for buffering refolding solutions including ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 mM, about 200 mM and at a variety of pH values, such as for example, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 mM, about 700 mM, about 1M.

Chaotropic agents are used to enhance refolding of OAS proteins, such chaotropic agents include: urea, guanidine HCl, thiourea, and the like, at concentrations such as for example about 0.05M, 0.1M, 0.25M, 0.5M, 1.0M, 2.0M, 3.0M, 4.0M, 5.0M, 6.0M, 7.0M, such as for example 8.0M and above including near saturation solutions. A variety of detergents are added to improve OAS protein refolding efficiency, such as Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.1% w/v, 0.2% w/v, 0.3% w/v, 0.4% w/v, 0.5% w/v, about 1% w/v, about 5% w/v, about 10% w/v, more than 10% w/v.

In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Chelating agents increase the half-life of thiol-reductants. Stabilizing agents including surfactants, sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added during refolding to stabilize OAS proteins.

Thiol-protective or reducing agents are added to prevent errant disulfide bond formation and to cleave inappropriate disulfide bond within the inclusion body, such thiol-protective groups including dithiothreitol (DTT), dithioerythritol (DTE),2-mercaptoethanol, 2-3-dimercaptopropanol, tributylphosphine (TBP), tris-carboxyethylphosphine (TCEP), thioglycolate, glutathione, and cysteine at concentrations of between 0.5 and 150 mM, such as for example, 1 mM, about 5 mM, about 10 mM, about 25 mM, about 50 mM, about 100 mM, about 150 mM.

OAS Protein API Purification, Concentration and Sterilization

In an exemplary embodiment, the properly refolded OAS protein-containing inclusion body preparations are filtered through a 0.45 micrometer membrane for clarification and loaded onto HiTrap Heparin HP® FPLC columns for initial capture and purification. Heparin columns bind approximately 4-5 mg of OAS protein per milliliter of resin. Heparin columns are pre-equilibrated with 50 mM NaH₂PO₄, 25 mM NaCl, 5% glycerol, 1 mM EDTA, 0.01% Tween-20®, 2 mM DTT, pH 6.8 before the application of refolded inclusion body preparations. OAS proteins bind efficiently to heparin columns. Once bound, immobilized OAS proteins are washed with two column volumes of 50 mM NaH₂PO₄, 25 mM NaCl, 5% glycerol, 1 mM EDTA, 0.01% Tween-20®, 2 mM DTT, pH 6.8 and eluted in a step gradient with 50 mM NaH₂PO₄, 1 M NaCl, 30% glycerol, 1 mM EDTA, 2 mM DTT, pH 6.8. Column chromatography is performed using fast protein liquid chromatography (FPLC) with commercially supplied columns or resins.

In another exemplary embodiment, HiTrap SP Fast Flow® columns are used when the conductance of the refolded protein preparation is below 6 mS/cm. In a still further embodiment, Cibacron Blue F3G-A (Blue Sepharose) resins are used that demonstrate a lower binding capacity for OAS proteins—approximately 1 mg/mL. In a still further exemplary embodiment, mixed mode resins (e.g. GE Healthcare's Capto MMC®) are used that bind OAS proteins at low affinity (e.g. <1 mg/mL resin). In a still further exemplary embodiment, Capto S and Phenyl HP columns are used for OAS protein capture from refolded inclusion body preparations.

An additional exemplary embodiment includes the use of carboxy-methyl (CM) cation exchange resins, which have been demonstrated to bind and capture OAS proteins from refold buffers. Such exemplary CM resins include but are not limited to: SP-FF (GE Healthcare), and CM Hyper-D (PALL). Further exemplary CM capture resins include but are not limited to: CM Sephadex C-25, CM Sephadex C-50, CM Sepharose High Performance, CM Sepharose Fast Flow (GE Healthcare), CM Ceramic HyperD F, CM Ceramic HyperZ (PALL), CM Sephadex® C-25, CM Sephadex®, C-50, CM Dextranomer C-25-120, CM Dextranomer C-50-120, CM-Cellulose (Sigma-Aldrich), Macro-Prep CM (BioRad), TSKgel CM-2SW, TSKgel CM-3SW, TSKgel CM-5PW (Tosoh), CM32, CM52 (Whatman), and Fractogel EMD COMM) (EMD Biosciences).

As one skilled in the art will recognize, numerous cation exchange resins are appropriate for the initial capture of OAS proteins from refolded, solubilized inclusion body preparations. Embodiments of appropriate cation exchange resin functional groups include: methyl sulfonate, sulfopropyl, carboxymethyl, sulfonic acid, carbonic acid, and carboxylic acid. Affinity resins that are derivatized with deoxyribonucleic acid or ribonucleic acid functional groups can also be used to practice the invention. Nicotinamide dye columns are also used to practice the invention. As one skilled in the art will recognize, a variety of column loading conditions and flow-rates are appropriate for a variety of industrial scales and applications.

Other embodiments include the use tangential flow filtration, diafiltration, dialysis, or gel filtration to allow for buffer exchange and concentration. One or more column steps may be substituted by selective precipitation with, for example, ammonium sulfate.

In one embodiment, buffers and buffer conditions, including buffer pH, can be altered to improve column binding capacities and efficiency. Buffer pH can also be altered to improve elution dynamics from the capture column. The following buffer components are used in column loading, wash and elution solutions: ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 mM, about 200 mM and at a variety of pH values, such as for example, lower than 5.0, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Refolding solutions can be diluted to reduce conductivity and improve column binding.

Detergents are added to prevent OAS protein aggregation and to limit non-specific protein interactions with the column matrix. A variety of detergent additives are envisioned as components of column loading, wash, and elution solutions including without limitation: Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.001% w/v, about 0.01% w/v, 0.02% w/v, about 0.05% w/v, 0.1% w/v, 0.2% w/v, 0.3% w/v, 0.4% w/v, 0.5% w/v, about 1% w/v, about 2% w/v, more than 2% w/v.

Reductants are used to prevent the formation of non-specific disulfide bonds. Column loading, wash, and elution buffers contain any of a number of reductants including without limitation: DTT, DTE, 2-mercaptoethanol, 2-3-dimercaptopropanol, TBP, TCEP, thioglycolate, glutathione, and cysteine at concentrations of between 0.5 and 150 mM, such as for example, 0.5 mM, 1 mM, 2 mM, 3 mM, 4 mM, about 5 mM, about 10 mM, about 25 mM, about 50 mM, about 100 mM, about 150 mM.

Salts are used to limit non-specific protein-protein interaction, to prevent protein aggregation, and to effect column elution from the cation exchange resin; typically used salts include: sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 mM, about 700 mM, about 1M, about 2M, about 3M. Low concentrations of chaotropic agents serve a similar role. Chelating and stabilizing agents are also included in column wash, and elution buffers as described elsewhere in the specification. All manner, combination and concentration of chelating and stabilizing agents are envisioned as components of column wash and elution buffers.

Hydrophobic interaction chromatography (HIC) is next used to purify OAS proteins away from E. coli host cell contaminants. HIC is an effective method for removing bacterial endotoxin and other pyrogens. In an exemplary embodiment, following elution of OAS protein-containing fractions from the initial cation exchange capture column, fractions are pooled and diluted 1:1 with 50 mM NaH₂PO₄, 300 mM NaCl, 20% glycerol, 1 mM EDTA, 2 mM DTT, pH 6.8 and adjusted to a final concentration of 1M ammonium sulfate. The OAS fractions are loaded onto a Phenyl HP HIC column at a protein density no greater than 7.5 mg/mL of resin. Columns are washed with three column volumes of a solution of 50 mM NaH₂PO₄, 300 mM NaCl, 1 M (NH₄)₂SO₄, 1 mM EDTA, 20% glycerol, 2 mM DTT, pH 6.8, followed by a step gradient to 40% of the following buffer: 50 mM NaH₂PO₄, 300 mM NaCl, 20% glycerol, 1 mM EDTA, 2 mM DTT, pH 6.8 for three column volumes. OAS protein containing fractions are eluted by a step gradient to 85% of the following buffer: 50 mM NaH₂PO₄, 300 mM NaCl, 20% glycerol, 1 mM EDTA, 2 mM DTT, pH 6.8 for three column volumes.

As one skilled in the art will recognize, a variety of column volumes and gradient functions will affect the same level of purity of OAS proteins following HIC. As one skilled in the art will further recognize, a number of salts and salt concentration are appropriate for column loading, wash and elution, with importance given to decreasing conductivity throughout the washing and elution steps.

In one embodiment, butyl, butyl S, octyl, or phenyl derivatized HIC columns are used for OAS capture and elution. Column loading, wash, and elution buffers are effectively formulated with one or more salts, buffers, stabilizing agents, detergents, reductants, and chelating agents at a variety of appropriate concentrations and pH's as described elsewhere in the specification.

Following HIC capture and elution, fractions containing OAS proteins are subjected to anion exchange chromatography to remove E. coli host cell contaminating pyrogens and nucleic acids. OAS containing fractions are diluted 1:5 with a solution composed of: 10 mM NaH₂PO₄, 20% glycerol, 1 mM EDTA, 2 mM DTT, pH 8. The pH of the resulting solution is adjusted to 8.0 by addition of HCl. The pH-adjusted sample is loaded onto a diethylaminoethyl (DEAE) FF column at a rate of 1.5 mL/minute and then washed with five column volumes of a solution composed of: 10 mM NaH₂PO₄, 20% glycerol, 1 mM EDTA, 2 mM DTT, pH 8. OAS proteins are found in the flow through. This step reduces endotoxin contamination to below 1 EU/mL.

As one skilled in the art will recognize, other anion exchange resins can be substituted for DEAE, including but not limited to those derivatized with quaternary ammonium and diethylaminopropyl groups. Other embodiments specifically for removing endotoxin contamination can be employed, including but not limited to the use of polymixin B columns.

Column loading and wash buffers are effectively formulated with one or more buffers, stabilizing agents, detergents, reductants, and chelating agents at a variety of appropriate concentrations and pH's as described elsewhere in the specification.

Following anion exchange chromatography to remove endotoxins, purified OAS proteins are concentrated by one of a variety of methods including cation exchange chromatography, ultrafiltration, or tangential flow filtration. Buffer exchanges are affected by gel filtration, tangential flow filtration/diafiltration, or ultrafiltration/diafiltration. Buffer exchange, protein concentration and sterilization result in an API suitable for inclusion into a pharmaceutical composition.

In one exemplary embodiment, the purified OAS protein is diluted to a conductivity of less than 6 mS/cm and the pH is adjusted to 6.8. The OAS protein is then bound to a cation exchange column, such as for example a HiTrap SP FF column, pre-equilibrated in a solution composed of 50 mM NaH₂PO₄, 25 mM NaCl, 20% glycerol, 1 mM EDTA, 2 mM DTT, pH 6.8. The bound OAS protein is washed with three column volumes of a solution composed of 50 mM NaH₂PO₄, 25 mM NaCl, 20% glycerol, 1 mM EDTA, 2 mM DTT, pH 6.8, and eluted with a step gradient to 70% of a solution composed of 50 mM NaH₂PO₄, 1 M NaCl, 30% glycerol, 2 mM DTT, 1 mM EDTA, pH 6.8. Purified OAS fractions are pooled and subjected to gel filtration for buffer exchange using a 2 mL/minute flow rate and a HiTrap desalting column. As one skilled in the art will recognize, any of a number of cation exchange and gel filtration columns will perform adequately for protein concentration and buffer exchange as described elsewhere in the specification. In other embodiments, purified OAS preparations are concentrated via ultrafiltration on Amicon polyethersulfone 10,000 membranes. Buffer exchange can be carried out by diafiltration. Final buffers are chosen based upon the required pharmaceutical composition for the API.

Exemplary Excipient Components for Purified OAS Proteins

OAS proteins are stabilized by excipients containing salts; solutions stable at 300 mM NaCl can begin to precipitate at 150 mM NaCl. For this reason excipient mixtures will favor these stabilizing salt concentrations, which could include but are not limited to sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate.

The addition of amino acid-based excipients such as arginine or glutamine has proven to be stabilizing to purified OAS proteins. The addition of 2% w/v arginine allows OAS proteins to be stable at 3 mg/mL. The addition of excipients such as glycerol is stabilizing to OAS polypeptides. For example, in one embodiment, a polypeptide has a maximum concentration with 10% glycerol (v/v) of 1 mg/mL; while at 40% glycerol, the OAS polypeptides are stable up to 12 mg/mL. Disaccharides such as sucrose have been found to be stabilizing at 10% w/v; other disaccharides including but not limited to maltose and trehalose are also used. Numerous stabilizing agents are appropriate for use as excipients components, including but not limited to: sugars and polyols (e.g. glycerol, sucrose, trehalose, glucose, lactose, inositol, mannitol, xylitol, ethylene glycol), surfactants (e.g. Tween-20®, Tween-80®), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules.

Antioxidants and preservatives are also used to ensure stability of purified OAS proteins during storage. Antioxidants, including but not limited to sodium citrate, may be stabilizing for long term storage of the OAS proteins. Preservatives, including but not limited to, benzyl alcohol may also be stabilizing to the polypeptides during storage and may be used in final excipient mixtures. An exemplary API intravenous formulation includes, but is not limited to: 10 mM sodium citrate, 270 mM sodium chloride, 7% w/v sucrose, pH 6.4.

Buffer Components

Bacterial cells and inclusion bodies containing recombinant OAS proteins are collected, washed, lysed, and solubilized under a variety of buffer and solution conditions. Furthermore, a variety of buffer and solution conditions are appropriate for each purification step in the entire manufacturing process leading to the production of a purified API. Without limiting the generality of the methods of the present invention, the methods disclosed herein include but are not limited to the use of alternate buffers, additives, and reagents as is known to one skilled in the art or as further exemplified in the following.

Buffers over a range of pKₐ values are used for buffering solutions including ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 mM, about 200 mM and at a variety of pH values, such as for example, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher.

Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 mM, about 700 mM, about 1M. Chaotropic agents are used to enhance refolding and stabilize OAS proteins, such chaotropic agents include: urea, guanidine HCl, thiourea, and the like, at concentrations such as for example about 0.05M, 0.1M,0.25M, 0.5M, 1.0M, 2.0M, 3.0M, 4.0M, 5.0M, 6.0M, 7.0M, such as for example 8.0M and above including near saturation solutions.

A variety of detergents are added to improve OAS protein refolding efficiency, to reduce protein aggregation, and to reduce the non-specific interaction of OAS proteins with solid supports, resins, tubes, containers, etc. Detergents also improve the stability of OAS proteins in solution. Exemplary detergent additives include Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.01%, about 0.02%, about 0.05%, about 0.07%, 0.1% w/v, 0.2% w/v, 0.3% w/v, 0.4% w/v, 0.5% w/v, about 1% w/v, about 5% w/v, about 10% w/v, more than 10% w/v.

In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Chelating agents increase the half-life of thiol-reductants. Stabilizing agents including sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added throughout the manufacturing process to stabilize OAS proteins.

Thiol-protective or reducing agents are added to prevent errant disulfide bond formation and to cleave inappropriate disulfide bonds within inclusion bodies, such thiol-protective groups include dithiothreitol (DTT), dithioerythritol (DTE),2-mercaptoethanol, 2-3-dimercaptopropanol, tributylphosphine (TBP), tris-carboxyethylphosphine (TCEP), thioglycolate, glutathione, and cysteine at concentrations of between 0.5 and 100 mM, such as for example, 1 mM, about 5 mM, about 10 mM, about 25 mM, about 50 mM, about 100 mM.

Exemplary Manufacturing Process Validation Methods

A number of biochemical methods are available to validate the purity and activity of in-process and final-stage purified OAS proteins manufactured according to the specification. The analytical methods include, but are not limited to, the following: quantification of protein concentration, measurement of protein purity, measurement of contaminants such as endotoxin, measurement of enzymatic activity (specific activity), and measurement of antiviral potency.

Quantification of Protein Concentration

The concentration of in-process and purified OAS proteins is measured by various assays known to one skilled in the art. One exemplary embodiment is a commercially available bicinchoninic acid (BCA) protein concentration assay kit such as the Reducing Agent Compatible BCA Protein Assay Kit from Pierce Biochemicals. A second exemplary embodiment is ultraviolet (UV) spectroscopy at a wavelength of 280 nm. In-process and purified proteins and their appropriate buffers are diluted in 6M Guanidine Hydrochloride (GuHCl) and the absorbance at 280 nm is recorded. Other appropriate diluents for the measurement of protein concentrations are envisioned. The concentration in mg/ml is calculated by multiplying the corrected absorbance (A280 sample-A280 background) by the appropriate extinction coefficient.

Measurement of Protein Purity

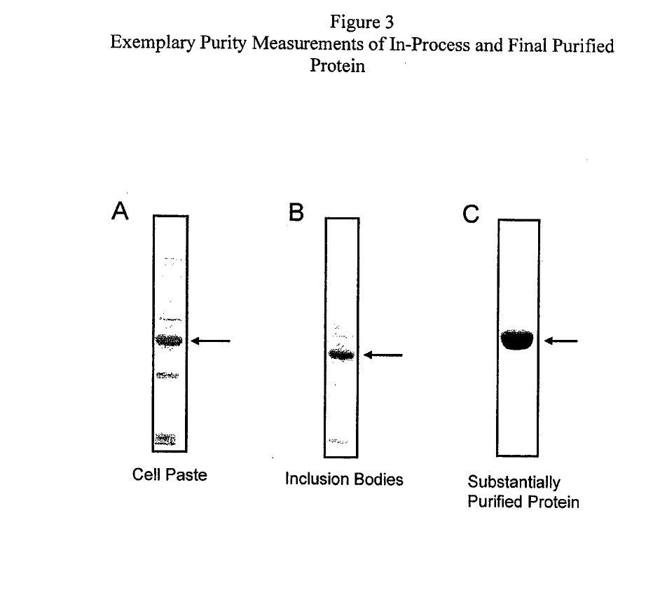

The purity of in-process and purified OAS proteins is measured by various analytical methods. One exemplary embodiment is Sodium Dodecyl Sulfate Polyacrylamide Gel Electrophoresis (SDS-PAGE) as shown in FIG. 3. In-process or purified OAS proteins are separated via SDS-PAGE and visualized using any appropriate method, including but not limited to Coomassie Brilliant Blue staining, silver staining, or western blot analysis with antibodies specific for OAS or contaminating proteins. The intensity of the specific OAS band and contaminating bands is compared by standard densitometry techniques known to one skilled in the arts. A second exemplary embodiment is size exclusion chromatography (SEC) using an appropriate chromatographic system. For example, in-process and purified OAS proteins are separated on size exclusion columns using either FPLC or HPLC chromatographic systems to ensure that the purified proteins are monomeric. A third exemplary embodiment is electrospray ionization mass spectrometry (ESI-MS), in which the sample is separated over an analytical column, ionized, and the mass to charge ratio detected by a mass spectrometer Impurities in the protein preparation are detected as different mass to charge signals. Further embodiments of a protein purity analysis method include the use of reverse-phase and ion-exchange HPLC.

Purity of OAS Protein Active Pharmaceutical Ingredients

OAS proteins in active pharmaceutical ingredients have purity as measured by SDS-PAGE of a single observed band that migrates in the same position as a reference standard without visible contamination by other protein bands. Purity is further measured by reverse-phase (RP)HPLC. OAS APIs have RP-HPLC purity of 70%, about 71%, about 72%, 73%, 74%, 75%, about 78%, about 80%, 81%, 82%, 83%, about 85%, about 87%, about 90%, about 92%, 93%, 94%, about 95%, greater than 95%. Purity is further measured by ion-exchange (IEX) HPLC. OAS APIs have IEX-HPLC purity of 70%, about 71%, about 72%, 73%, 74%, 75%, about 78%, about 80%, 81%, 82%, 83%, about 85%, about 87%, about 90%, about 92%, 93%, 94%, about 95%, greater than 95%. Endotoxin levels in OAS protein APIs are less than 20 EU/mg, 19EU/mg, 18EU/mg, 17EU/mg, about 15EU/mg, 14EU/mg, 13EU/mg, about 12EU/mg, about 10EU/mg, about 8EU/mg, about less than 5EU/mg, about 4EU/mg, 3EU/mg, 2EU/mg, less than 1EU/mg, or less than the limit of detection of the assay such that the total endotoxin burden administered to a human subject per dose of API dose not exceed 5EU/kg/hour. Bioburden levels in OAS protein APIs are less than 100 colony-forming units (CFU) per milliliter, less than 80 CFU/ml, less than about 50 CFU/ml, less than 25 CFU/mL, less than 10 CFU/mL, less than about 5EU/mL, less than about 1EU/mL, or less than the limit of detection of the assay.

Host cell DNA contamination in OAS protein APIs is less than 500 pg/mg, less than about 200 pg/mg, less than about 150 pg/mg, less than about 100 pg/mg, less than about 80 pg/mg, less than about 60 pg/mg, less than about 40 pg/mg, less than about 20 pg/mg, less than about 10 pg/mg, 9 pg/mg, 8 pg/mg, 7 pg/mg, 6 pg/mg, less than 5 pg/mg, or less than the limit of detection of the assay. Host cell protein contamination in OAS protein APIs is less than 250 ng/mg, less than about 150 ng/mg, less than about 100 ng/mg, less than about 80 ng/mg, less than about 60 ng/mg, less than about 40 ng/mg, less than about 20 ng/mg, less than about 10 ng/mg, 9 ng/mg, 8 ng/mg, 7 ng/mg, 6 ng/mg, less than 5 ng/mg, or less than the limit of detection of the assay. Aggregate amounts of OAS protein in APIs by SEC-HPLC are less than 10% of total protein, less than about 9%, less than about 7%, less than about 5%, less than about 2%, or less than 1%. Antibiotic contamination in OAS protein APIs are less than 1000 ng/mg, less than about 500 ng/mg, less than about 250 ng/mg, less than about 100 ng/mg, or less than the limit of detection of the assay. IPTG contamination in OAS protein APIs are less than about 250 ng/mg, less than about 100 ng/mg, less than about 75 ng/mg, less than about 50 ng/mg, less than about 25 ng/mg, or less than the limit of detection of the assay.

Measurement of Contaminants

Assessment of in-process and final purified protein purity includes a measure of contaminants, including but not limited to host cell proteins and pyrogens such as endotoxin. Contamination with other proteins, including host cell proteins, can be assessed using the same techniques described in the section above (Measurement of Protein Purity) as well as standard enzyme-linked immunosorbent assay methods. One exemplary embodiment of pyrogen testing is the Limulus Amoebocyte Lysate (LAL) endotoxin assay. Various commercially available assay kits are available that utilize a modified LAL and synthetic color-producing substrate to detect endotoxin presence. As one skilled in the art will recognize, numerous methods for measuring host cell DNA and protein contamination, pyrogen contamination and endotoxin contamination in manufactured APIs are in common use in the commercial manufacture of drug products, and the invention is not limited by the use of any specific method.

Measurement of OAS Enzymatic Activity

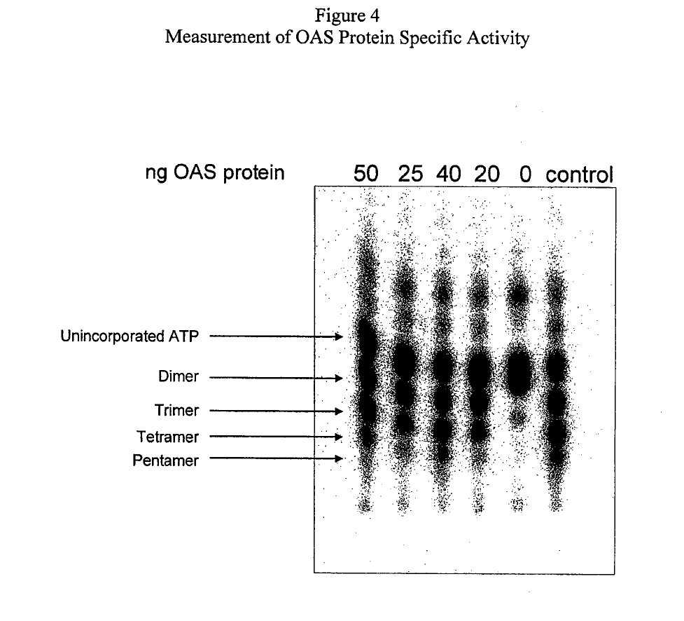

The oligoadenylate synthetase activities of the in-process samples and final purified OAS proteins manufactured as per this invention are measured according to previously published methods (Justesen, J., et al. Nuc Acids Res. 8:3073-3085, 1980). Briefly, protein is activated with 200 μg/ml polyinosinic:polycytidylic acid (polyI:C) in buffer containing 20 mM Tris-HCl, pH 7.8, 50 mM Mg(OAc)₂, 1 mM DTT, 0.2 mM EDTA, 2.5 mM ATP, α[³²P]ATP, 0.5 mg/ml BSA, and 10% glycerol. The reaction proceeds at 37° C. for 30 minutes to 24 hours and is terminated by heating to 90° C. for 3 minutes. 2-4 μl of the reaction mixture is spotted onto a polyethylenimine PEI-cellulose thin layer plate (TLC). After drying, the plate is developed with 0.4 M Tris-HCl, 30 mM MgCl₂, pH 8.7. The plate is dried and visualized by phosphorimager analysis; a representative TLC image is shown in FIG. 4. Alternatively, the reaction mixture can be further incubated with 0.05 U/μl calf intestinal phosphatase to remove the terminal phosphate. Thin layer chromatographic separation is achieved using a 0.76 M KH₂PO₄, pH 3.6 developing buffer system. The plate is then dried and visualized by phosphorimager analysis.

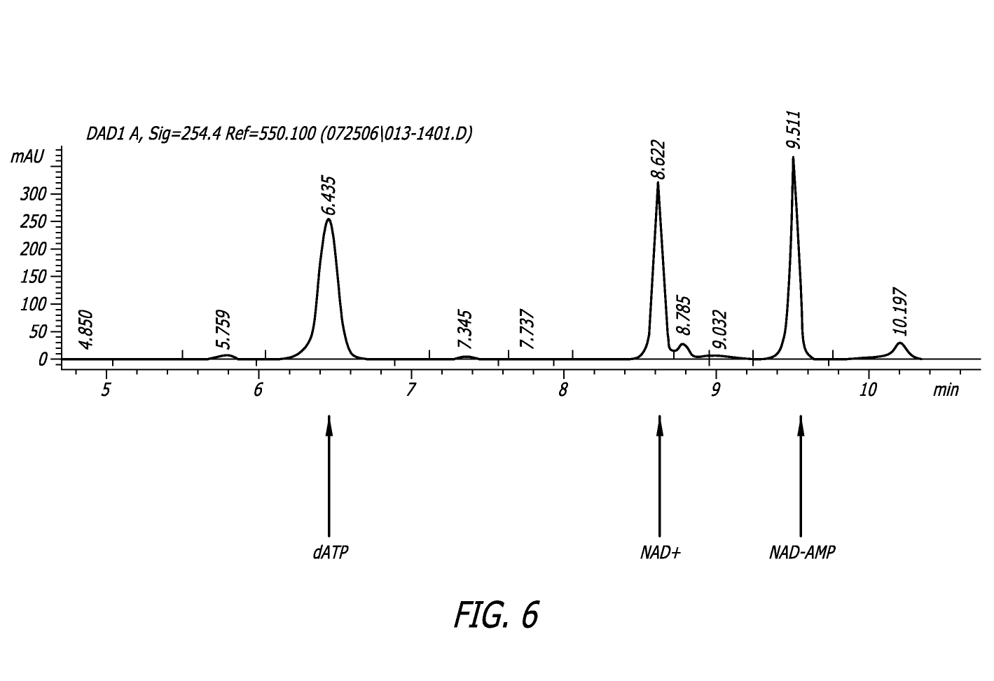

A second exemplary embodiment of a method to assess enzymatic activity is to measure the catalysis of NAD-AMP by OAS proteins from the substrates β-Nicotinamide adenine dinucleotide (NAD) and dATP. Different concentrations of protein are mixed with 2 mM NAD, 2 mM dATP, 4 mM Tris pH 7.8, 4 mM Mg(OAc)₂, 0.2 mM DTT, 0.04 mM EDTA, 0.1 mg/ml BSA and 0.05 mg/ml polyI:C. The sample is incubated at 37° C. for 20 min, and the reaction is stopped by heating at 80° C. for 2 min or by column or membrane purification of reaction products. The sample is spun down and an aliquot taken and diluted 1:1 with an appropriate mobile phase buffer. The analytes are separated via C18 column chromatography on an HPLC. An example is shown in FIG. 6. Area under the curve analysis of the peaks is used to calculate the percent conversion NAD and dATP to the NAD-AMP product.

Measurement of Antiviral Activity of OAS Polypeptides

Potency of in-process and final purified OAS proteins are demonstrated using a variety of cell culture antiviral assays. One exemplary embodiment of antiviral activity is the ability of the manufactured proteins to protect cultured cells from cytotoxicity induced by the murine encephalomyocarditis virus (EMCV, ATCC strain VR-129B). Human Huh7 hepatoma cells are seeded at a density of 1×10⁴ cells/well in 96 well culture plates and incubated overnight in complete medium (DMEM containing 10% fetal bovine serum). The following morning, the media is replaced with complete medium containing 0-10 μM protein or equivalent amounts of protein dilution buffer. When desired, alpha-interferon is added at a concentration of 100 IU/ml. Cells are pretreated for 2-8 hours preceding viral infection. After pretreatment, an equal volume of medium containing dilutions of EMC virus in complete medium is added to the wells.

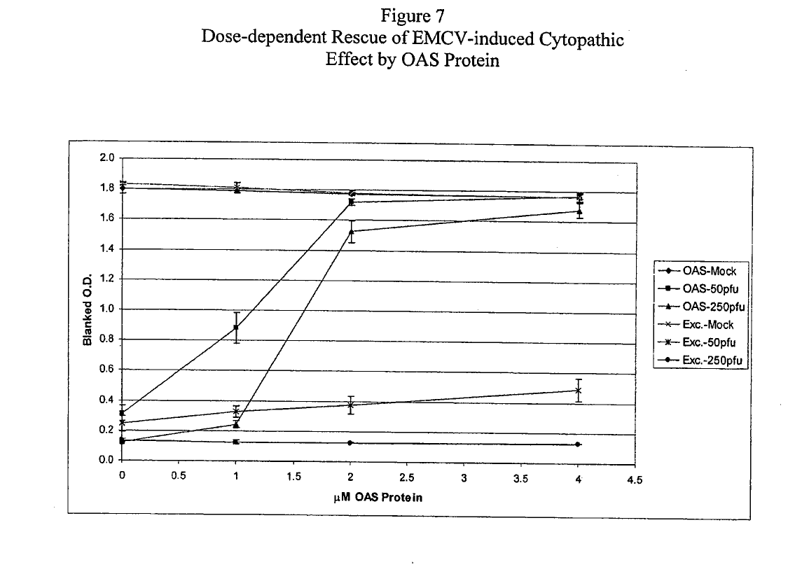

In the experiments described herein, a range of 50-250 plaque forming units (pfu) is added per well. Viral infection is allowed to proceed overnight (approximately 18 hours), and the proportion of viable cells is calculated using any available cell viability or cytotoxicity reagents. The results described herein are obtained using a cell viability assay that measures conversion of a tetrazolium compound [3-(4,5-dimethyl-2-yl)-5-(3-carboxymethoxyphenyl)-2-(4-sulfophenyl)-2H-tetrazolium, inner salt; MTS] to a colored formazan compound in viable cells. The conversion of MTS to formazan is detected in a 96-well plate reader at an absorbance of 492 nm. The resulting optical densities either are plotted directly (e.g. FIG. 7) to estimate cell viability or are normalized by control-treated samples to calculate a percentage of viable cells after treatment.

Other in vitro virus infection models include but are not limited to flaviviruses such as HCV, bovine diarrheal virus, West Nile Virus, and GBV-C virus, and other RNA viruses such as respiratory syncytial virus, and the HCV replicon systems (e.g. Blight, K. J., et al. 2002. J. Virology, 76:13001-13014). Any appropriate cultured cell competent for viral replication can be utilized in the antiviral assays.

### EXAMPLES

### Example 1

### Exemplary Polynucleotides Useful in the Implementation of the Present Invention

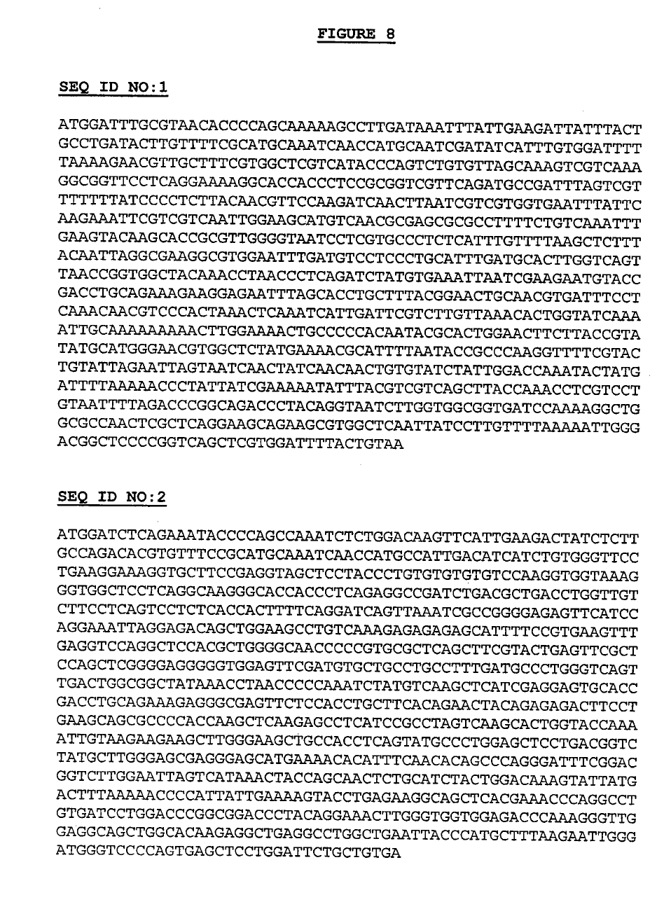

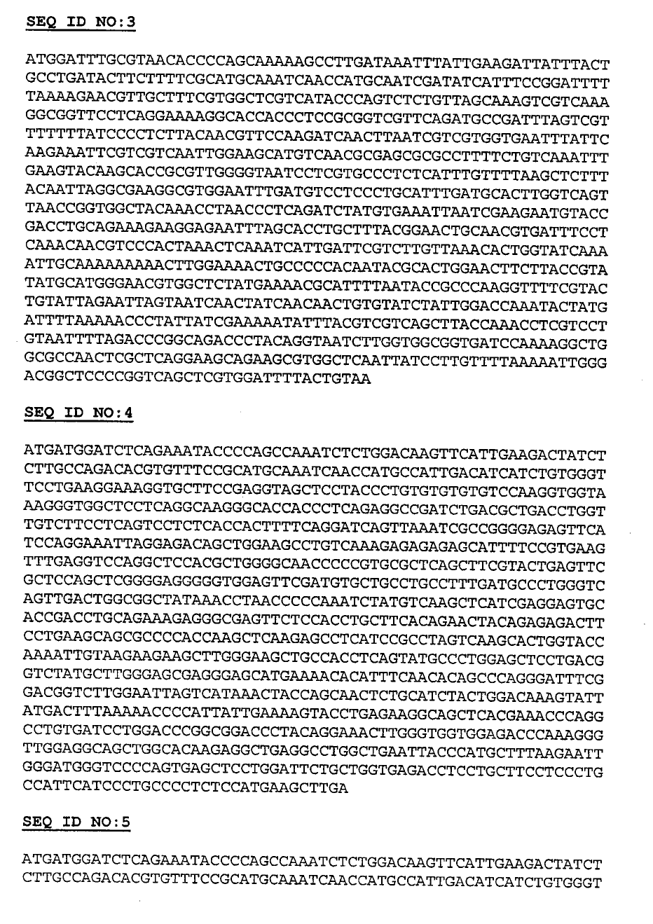

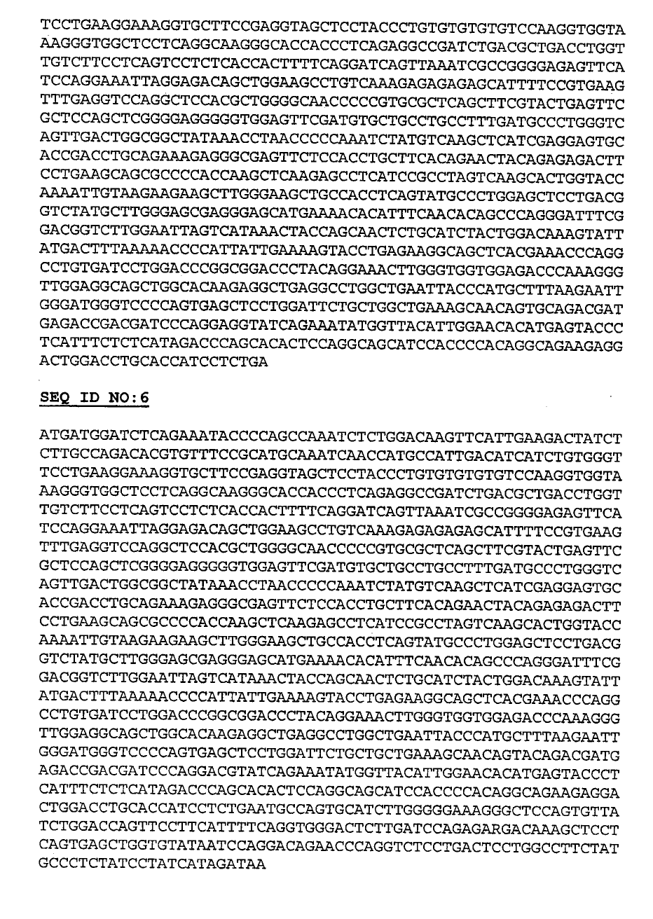

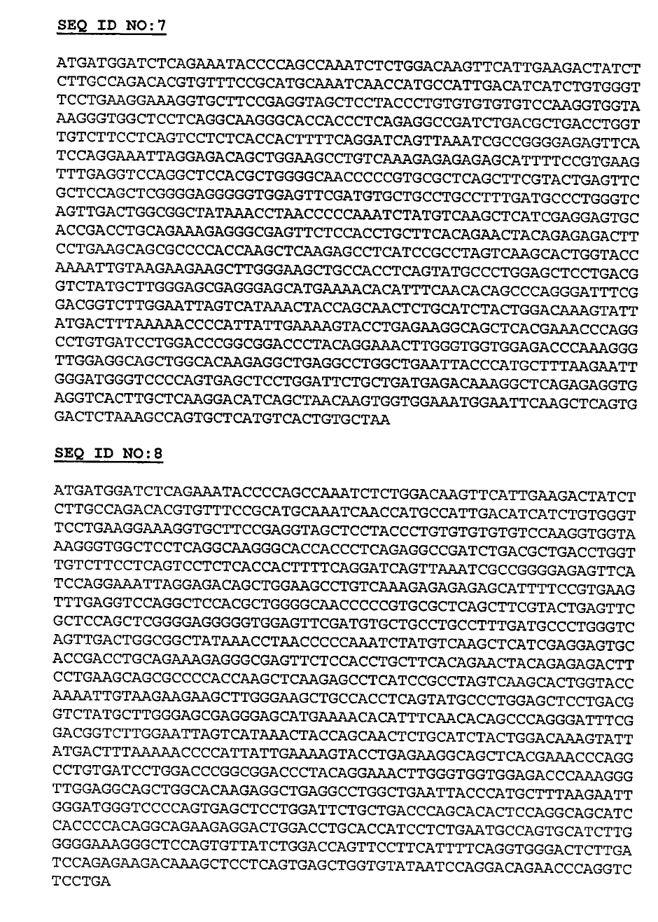

Each of the polynucleotides described by the sequences of FIG. 8 (SEQ ID NO:1-8) is useful in an implementation of the present invention. In particular, these polynucleotides encode variant polypeptides of the OAS1 type that are useful products of the process of the present invention. Each of these polynucleotides, for example, is used as a component of an expression vector (the construction of which is as described elsewhere in the present invention and or known to those skilled in the art) that is used to express the desired polypeptide or protein in a suitable expression system.

### Example 2

### Exemplary Polypeptides that are Produced by Implementation of the Present Invention

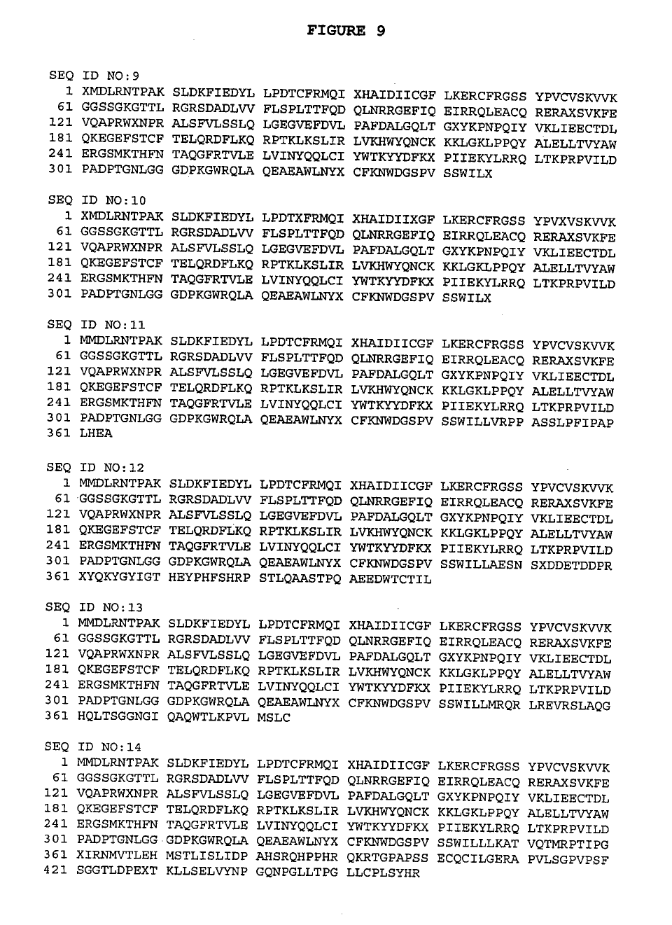

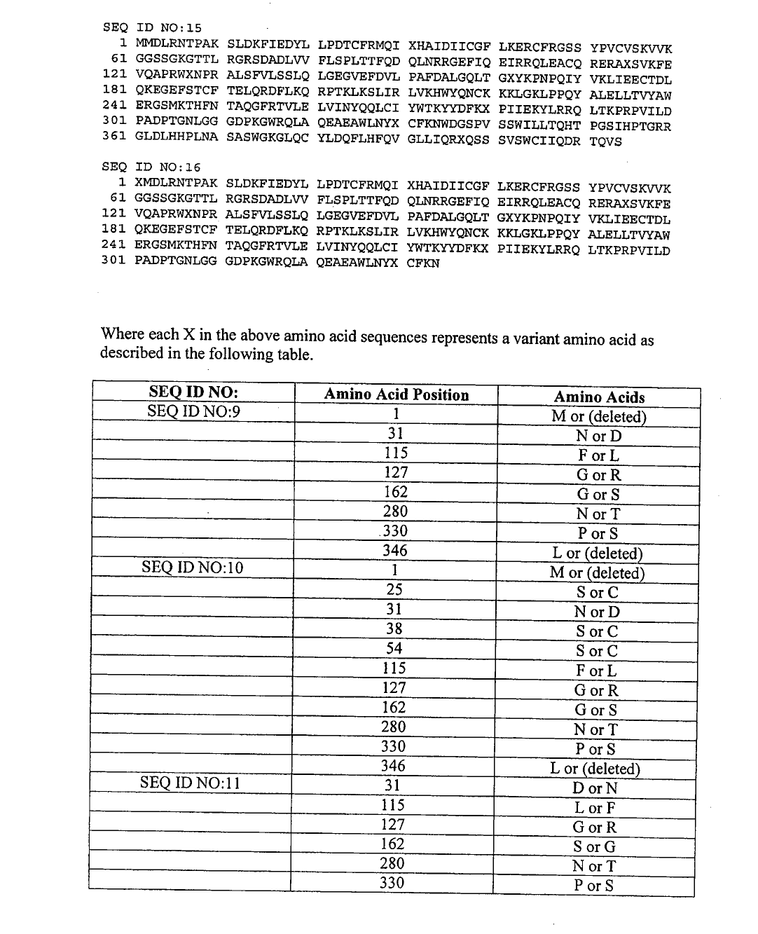

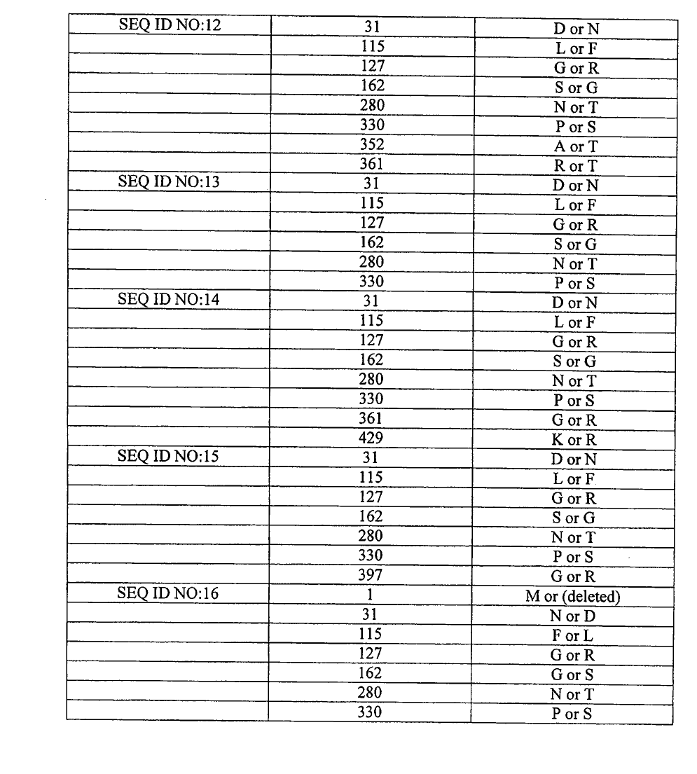

Each of the polypeptides described by the sequences of FIG. 9 (SEQ ID NO:9-16) is a useful product that is obtained by an implementation of the present invention. These polypeptides are variants of the OAS1 type, a class of proteins which themselves have utility as described elsewhere in the present invention, and therefore demonstrate the utility of the present invention.

### Example 3

### Exemplary Chromatograms from the Manufacturing Process

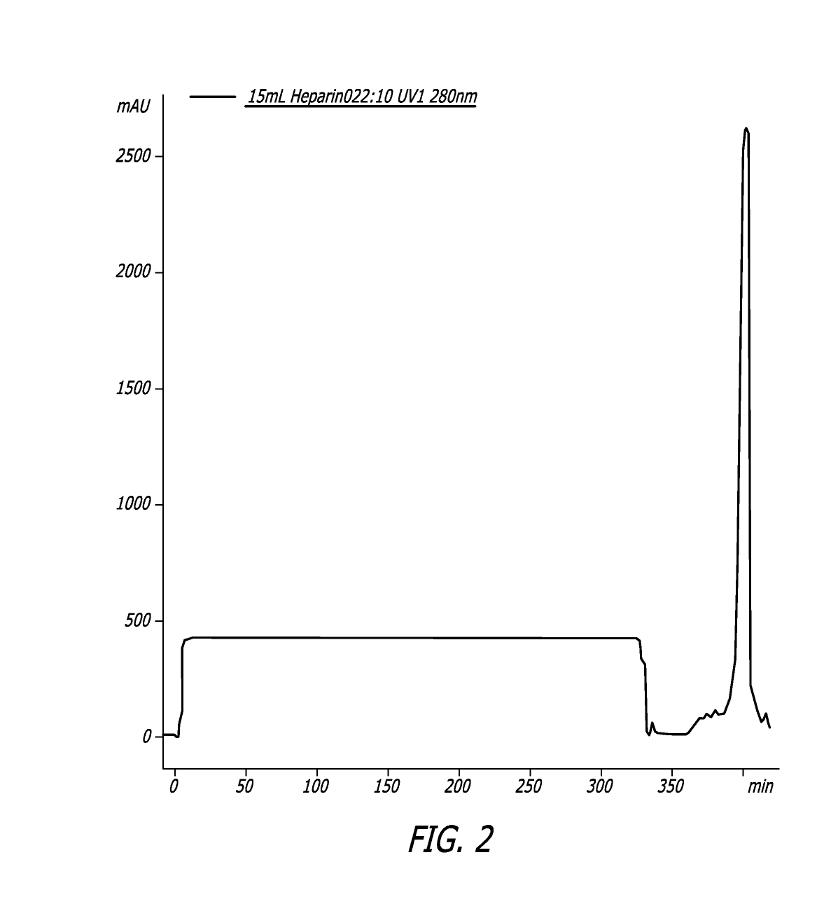

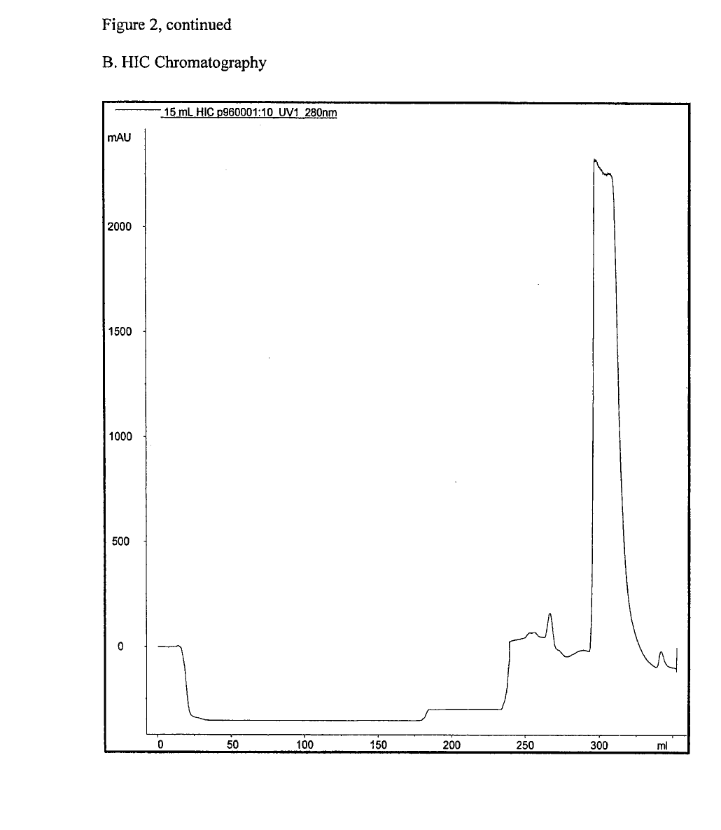

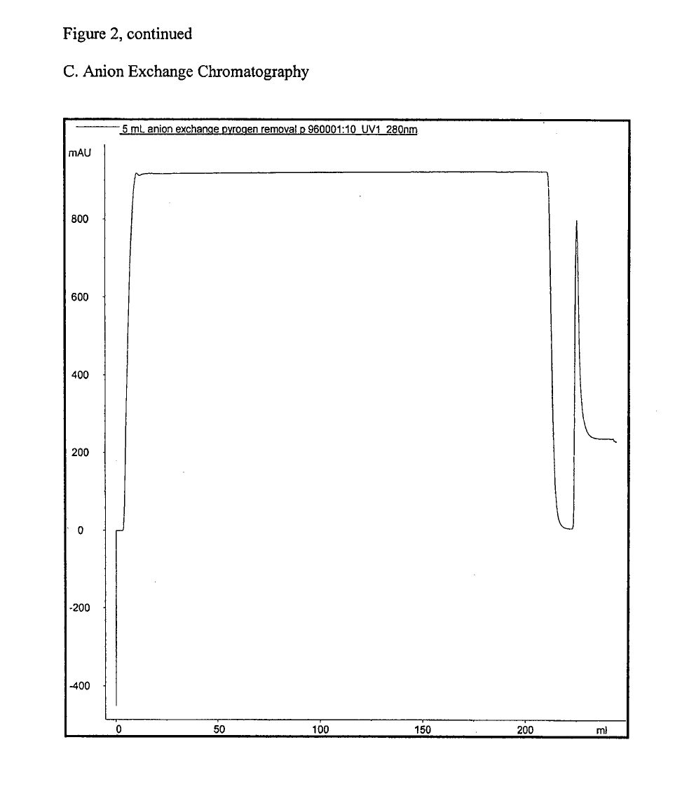

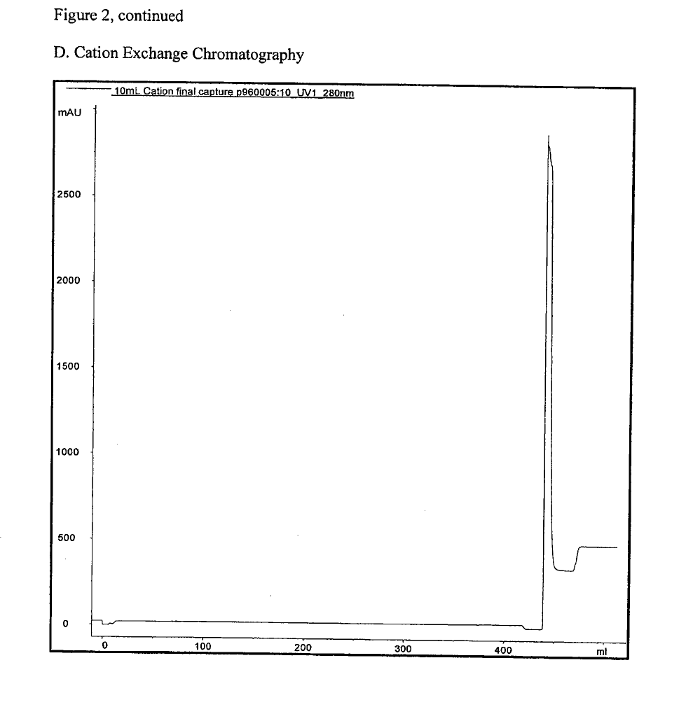

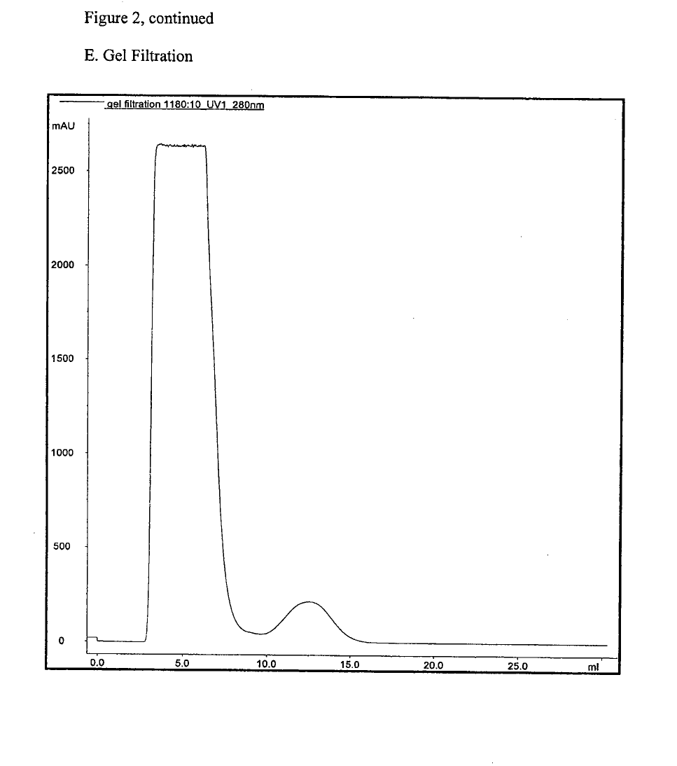

Representative chromatograms for steps 4-8 of the manufacturing process are shown in FIG. 2, Panels A-E. For each column, the ultraviolet (UV) absorbance at 280 nm was monitored during protein fraction collection. Panel (A) shows an example of a typical peak observed during Step 4 of the process, initial capture of refolded protein. In this example, capture of the OAS protein occurred on a HiTrap Heparin column. A refolded inclusion body preparation of ˜20% purity was loaded onto the heparin column in 50 mM NaH₂PO₄, 25 mM NaCl, 10% glycerol, 1 mM EDTA, 0.01% Tween-20®, 2 mM DTT, pH 6.8 at room temperature. The protein was eluted using a gradient from a buffer consisting of 50 mM NaH₂PO₄, 25 mM NaCl, 10% glycerol, 1 mM EDTA, 0.01% Tween-20®, 2 mM DTT, pH 6.8 to 100% of a buffer consisting of 50 mM NaH₂PO₄, 1 M NaCl, 30% glycerol, 2 mM DTT, 1 mM EDTA, pH 6.8, and peak fractions were collected.

In Step 5, hydrophobic interaction column (HIC) purification is utilized to remove endotoxin and further purify the protein. Panel (B) shows a representative chromatogram for Step 5 of the process. In this example, pooled fractions from the heparin column purification step were diluted 1:1 with a buffer consisting of 50 mM NaH₂PO₄, 300 mM NaCl, 20% glycerol, 1 mM EDTA, 2 mM DTT, pH 6.8. The diluted protein was adjusted to 1 M (NH₄)₂SO₄ and loaded onto a Phenyl FF HP column. The column was washed with a buffer consisting of 50 mM NaH₂PO₄, 300 mM NaCl, 20% glycerol, 1 mM EDTA, 2 mM DTT, 1 M (NH₄)₂SO₄, pH 6.8. The protein was eluted with the following three step gradients: (i) to 40% of a buffer consisting of 50 mM NaH₂PO₄, 300 mM NaCl, 20% glycerol, 2 mM DTT, 1 mM EDTA, pH 6.8, (ii) to 85% of a buffer consisting of 50 mM NaH₂PO₄, 300 mM NaCl, 20% glycerol, 2 mM DTT, 1 mM EDTA, pH 6.8, and (iii) 100% of a buffer consisting of 50 mM NaH₂PO₄, 300 mM NaCl, 20% glycerol, 2 mM DTT, 1 mM EDTA, pH 6.8.

Step 6 of the process described herein utilizes anion exchange chromatography to remove contaminating pyrogens. A representative chromatogram for the anion exchange chromatography is shown in Panel (C) of FIG. 2. In this example, anion exchange chromatography of OAS utilized a DEAE column and allowed flow through column chromatography for endotoxin (pyrogen) removal. Pooled HIC fractions were diluted (1:5) with a buffer consisting of 10 mM NaH₂PO₄, 20% glycerol, 2 mM DTT, 1 mM EDTA, pH 8, and loaded onto the column in the same buffer. The protein is collected in the flow through fraction. The column is washed by a step gradient to 100% of a buffer consisting of 50 mM NaH₂PO₄, 1 M NaCl, 30% glycerol, 2 mM DTT, pH 6.8.

Step 7 of the process described herein provides for concentration of OAS protein via cation exchange chromatography, ultrafiltration, or other equivalent methods. In this example, cation exchange chromatography was used to concentrate the protein solution; a representative chromatogram of the peak fraction is shown in Panel (D) of FIG. 2. Pooled anion exchange fractions were diluted (1:1) with 10 mM NaH₂PO₄, 20% glycerol, 2 mm DTT, 1 mM EDTA, pH 6.8, and loaded onto an HiTrap SPFF cation exchange column. Protein was eluted using a step gradient to 70% of a buffer consisting of 50 mM NaH₂PO₄, 1 M NaCl, 30% glycerol, 2 mM DTT, 1 mM EDTA, pH 6.8.

Panel (E) shows an example of Step 8 of the OAS purification process, gel filtration to exchange the protein into the desired buffer. Fractions from the cation exchange chromatography step were loaded onto a gel filtration column and exchanged into buffer consisting of 50 mM NaH₂PO₄, 300 mM NaCl, 25% glycerol, 1 mM EDTA, 2 mM DTT, pH 6.8.

### Example 4

### Electrophoretic Analysis of in Process Manufacturing Samples

FIG. 3 provides an example of the purification of an OAS protein resulting from the manufacturing process described in the specification. Expression and purity of OAS at various purification stages were analyzed by SDS-PAGE gel electrophoresis and SimplyBlue™ SafeStain (Invitrogen Corporation) visualization (equivalent to Coomasie Blue visualization). The band representing OAS is indicated with an arrow in each panel. Fermentation, inclusion body preparation and purification were performed as described in the specification. (A) Fermentation of OAS-expressing E. coli yielded a prominent protein band (˜10% of total cellular protein) in the cell paste. (B) Purification and solubilization of inclusion bodies in Step 2 of the manufacturing process demonstrates significant enrichment of OAS in inclusion bodies. (C) Final purification of OAS in Step 8 resulted in a substantially pure protein product.

### Example 5

### Measurement of Specific Activity of in Process and Substantially Pure OAS Protein Samples

The manufacturing process described in the specification results in the production of highly active protein. FIG. 4 and TABLE 1 provide examples of an OAS activity assay used to measure specific activities of various in-process and final API OAS protein samples (TABLE 1). FIG. 4 represents typical results obtained when using a radioactive assay to assess OAS activity. Different concentrations of OAS were mixed in 4 mM Tris-HCl, pH 7.8, 10 mM Mg(OAc)₂, 2.5 mM ATP, 200 μg/ml poly I:C, 0.1 mg/ml BSA, 2% glycerol, 0.2 mM DTT, 0.04M EDTA and α[32P]ATP and incubated for 30 min at 37° C. Samples were boiled to stop the enzymatic reaction and 4 μl applied onto PEI-plates for thin-layer chromatography. The plates were developed in 0.4 mM Tris-HCl, pH 8.65, 30 mM MgCl₂ and analyzed using a phosphorimager. The results are expressed in nmoles ATP incorporated per minute per mg OAS protein. In this example, the formation of oligoadenylates from 50 ng, 25 ng, 40 ng, 20 ng, 0 ng (blank), and a positive control (1 μg) of protein, are represented. Spots representing unincorporated ATP and oligoadenylate dimers, trimers, tetramers, and pentamers are indicated on the image.

TABLE 1 shows representative specific activities of OAS at different steps in the manufacturing process to demonstrate consistency in activity. Aliquots of protein were collected after the indicated step in the manufacturing process and tested over a range of concentrations to determine the specific activity. In this example, the final purified product had a specific activity of 17.6 nmoles ATP incorporated per minute per milligram protein.

TABLE 1
Exemplary Specific Activity of In-Process and Purified OAS Proteins

| | | Specific Activity |
|---|---|---|
| | | (nmoles ATP |
| | | incorporated/min/mg |
| Manufacturing Step | Sample | protein) |
| 3 | Refolded protein | 16.0 |
| 4 | Heparin column | 16.4 |
| 5 | Hydrophobic Interaction | 10.0 |
| | column | |
| 9 | Final product | 17.6 |

### Example 6

### Parameters Affecting OAS Protein Refolding Efficiency

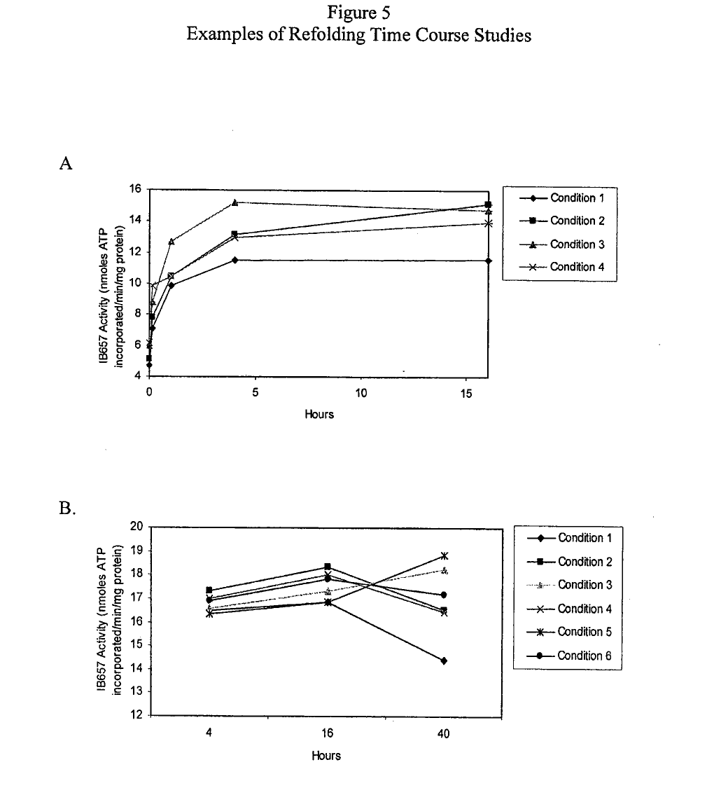

FIG. 5 describes the effect of varying conditions on OAS refolding efficiency. FIG. 5 demonstrates the effect of different buffers, reducing agents, pH, time, and temperature on refolding efficiency and subsequent specific activity of the protein. Significant differences in specific activity of the refolded inclusion body preparations are apparent depending on the refolding conditions tested.

Panel (A) shows the specific activities of proteins refolded in a set of buffers designed to test the effect of time, detergent, salt or arginine concentration on refolding. Inclusion bodies were solubilized in 50 mM NaH₂PO₄, 6 M guanidine HCl, 100 mM DTT, pH 8. Protein was refolded by pulse dilution into a stirring solution of refolding buffer at room temperature. The refolding buffers tested included addition of: Condition 1, 50 mM NaH₂PO₄, pH 6.8, 10% glycerol, 1.2 mM DTT, 1% Tween-20®, 25 mM NaCl; Condition 2, 50 mM NaH₂PO₄, pH 6.8, 10% glycerol, 1.2 mM DTT, 0.5% Tween-20®, 25 mM NaCl; Condition 3, 50 mM NaH₂PO₄, pH 6.8, 10% glycerol, 1.2 mM DTT, 0.5% Tween-20®, 500 mM NaCl; and Condition 4, 50 mM NaH₂PO₄, pH 6.8, 10% glycerol, 1.2 mM DTT, 0.5% Tween-20®, 500 mM NaCl, 300 mM Arginine. 50 ng of refolded protein were tested for OAS activity after 0, 10 minutes, 1, 4, and 16 hours. Conditions 2-4 resulted in slightly more active protein than Condition 1 in this experiment.

Panel (B) shows the results of a separate set of experiments undertaken to test the effect of time, reductant, pH, temperature, presence of bovine serum albumin (BSA) and presence of guanidine hydrochloride on refolding. Specific activities of refolded proteins were measured after 4, 16, and 40 hours. The refolding conditions used included:

Condition 1: Solubilized inclusion body solution was added to 50 mM NaH₂PO₄, pH 6.8, 10% glycerol, 1 mM EDTA, 0.05% Tween-20, 0.3 M NaCl, 20 mM DTT, and rotated at room temperature. After 4 hours, additional DTT to 20 mM was added.

Condition 2: Solubilized inclusion body solution was added to 50 mM NaH₂PO₄, pH 6.8, 10% glycerol, 1 mM EDTA, 0.05% Tween-20, 0.3 M NaCl, 1.8 μM 2-mercaptoethanol (BME), and rotated at room temperature. After 4 hours, additional BME to 5 mM was added.

Condition 3: A solution of 20 mM NaH₂PO₄, pH 6.8, 10% glycerol, 1 mM EDTA, 0.05% Tween-20, 0.3 M NaCl, 20 mM DTT, was chilled for 1 hour on ice. Solubilized inclusion body solution was then added to the cold solution and rotated at 4° C. for the first 16 hours. The solution was then rotated at room temperature from 16-40 hours. After 4 hours additional DTT to 20 mM was added.

Condition 4: Solubilized inclusion body solution was added to 20 mM NaH₂PO₄, pH 8.0, 10% glycerol, 1 mM EDTA, 0.05% Tween-20, 0.3 M NaCl, 20 mM DTT and rotated at room temperature. After 4 hours, additional DTT to 50 mM was added.

Condition 5: Solubilized inclusion body solution was added to 20 mM NaH₂PO₄, 10% glycerol, 1 mM EDTA, 0.05% Tween-20, 0.3 M GuHCl, 20 mM DTT and rotated at room temperature. After 4 hours, additional DTT to 50 mM was added.

Condition 6: Solubilized inclusion body solution was added to 50 mM NaH₂PO₄, pH 6.8, 10% glycerol, 1 mM EDTA, 0.05% Tween-20, 0.3 M NaCl, 50 mM DTT, 0.01% BSA, and rotated at room temperature. After 4 hours, additional DTT to 50 mM was added.

### Example 7

### Exemplary HPLC-Based Assay for Measuring OAS Specific Activity

FIG. 6 shows a sample HPLC chromatogram of the NAD-dATP assay demonstrating the production of NAD-AMP by OAS protein purified by the method of the specification. Different concentrations of protein were mixed with 2 mM NAD, 2 mM dATP, 4 mM Tris, pH 7.8, 4 mM Mg(OAc)₂, 0.2 mM DTT, 0.04 mM EDTA, 0.1 mg/ml BSA and 0.05 mg/ml poly I:C. The sample was incubated at 37° C. for 20 min and the reaction stopped by heating at 80° C. for 2 min. The sample was spun down and an aliquot taken and diluted 1:1 with the mobile phase buffer. For HPLC analysis, 10 μl of the diluted sample were loaded onto a Supelco Ascentis C18 column in mobile phase consisting of 50 mM NH₄H₂PO₄, pH 7, using a flow rate of 1.5 ml/min. The analytes were eluted with a gradient from 5-60% of MeOH:Water (1:1) (v/v). Area under the curve analysis of the peaks was used to calculate percent conversion to the NAD-AMP product. dATP, NAD and NAD-AMP eluted at 6.435, 8.622, and 9.511 min, respectively.

### Example 8

### Exemplary Methods for Measuring OAS Protein Antiviral Activity

OAS protein purified using the methods described in the specification is antiviral. FIG. 7 shows typical results obtained after assessing the antiviral potency of purified OAS proteins. Monolayers (˜85% confluent) of Huh7 human hepatoma cells were pretreated for 8 hours with the indicated doses of OAS protein or equivalent excipient (exc.) concentrations. After 8 hours, media containing 0 (mock), 50, or 250 plaque forming units (pfu) of the cytopathic murine encephalomyocarditis virus (EMCV) were added to the wells. Virus infections were allowed to proceed for 18 hours. Rescue from the EMCV-induced cytopathic effect was measured using the conversion of a tetrazolium compound [3-(4,5-dimethyl-2-yl)-5-(3-carboxymethoxyphenyl)-2-(4-sulfophenyl)-2H-tetrazolium, inner salt; MTS] to a colored formazan compound in viable cells. The conversion of MTS to formazan was detected in a 96-well plate reader at 492 nm, and the resulting optical densities were plotted directly to estimate cell viability (higher O.D. indicates more viable cells).

### Example 9

### Second Exemplary HPLC-Based Assay for Measuring OAS Specific Activity

Different concentrations of protein were mixed with 2.5 mM NAD, 2.5 mM dATP, 20 mM Tris, pH 7.8, 25 mM Mg(OAc)₂, 2 mM DTT, 1 mM EDTA and 0.25 mg/ml poly I:C. Each sample was incubated at 37° C. for 20 min and the reaction stopped by cooling to 4° C. for at least 10 min. The samples were spun down and a 50 μl aliquot was taken for HPLC analysis. The 50 μl aliquots were individually loaded onto a 25 cm Supelco Ascentis C18 column (5 micron beads) for HPLC separation. The analytes were eluted using a non-linear gradient of 0-60% MeOH: mobile phase buffer (50 mM NH₄H₂PO₄, pH 7) (1:1) (v/v) using a flow rate of 1.5 ml/min. Analytes and product were detected at 254 nm. Area under the curve analysis of the peaks was used to calculate percent conversion to the NAD-AMP product. dATP, NAD and NAD-AMP eluted at 4.1, 4.7, and 5.5 min, respectively.

### Example 10

### Exemplary Intravenous Formulation

The OAS proteins were formulated in a buffered solution suitable for intravenous administration to a mammal that included the following components: 9.9 mg/mL OAS protein, 10 mM histidine, 500 mM NaCl, 5% manitol, pH 5.5.

### Example 11

### Exemplary OAS Protein Drug GMP Release Specification

Drug GMP Release Specifications

| | | | Laboratory-scale Drug |
|---|---|---|---|
| Method | Attribute | Acceptance Criteria | Lot^{1,2} |
| Drug Concentration by | Strength | >10 mg/ml | Conforms |
| A280 | | | |
| Activity Assay | Potency | 50 to 150% of reference | ND |
| | | standard | |
| IEX-HPLC | Purity | ≧95% main peak | >94% |
| Size exclusion HPLC | Purity | ≧95% main peak | 99% |
| | | <1% aggregate/high MW | |
| | | species | |
| Reversed phase HPLC | Purity | ≧95% main peak | ND |
| Osmolality | Purity | Isotonic | ND |
| Endotoxin | Safety | ≦0.5 EU/mg | <0.1 EU/mg |
| Bioburden | Safety | ≦1 CFU/mL | ND |
| SDS-PAGE reduced | Identity | Conforms to reference standard | >99% pure |
| SDS-PAGE non- | Identity | Conforms to reference standard | Conforms |
| reduced | | | |
| Isoelectric focusing | Identity | Conforms to reference standard | ND |
| Western blot | Identity | Conforms to reference standard | Conforms |
| N terminal sequencing | Identity | Conforms to reference standard | Conforms |
| | | | (100%) |
| Whole mass MS | Identity | Conforms to reference standard | Conforms |
| | | +/−2 kD | |
| Residual host cell | Impurity | ≦100 ng/mg | <1 ng/mL |
| protein | | | |
| Residual host cell DNA | Impurity | ≦100 pg/dose | ND |
| Residual kanamycin | Impurity | Not detectable | ND |
| Residual IPTG | Impurity | Not detectable | ND |
| Color and appearance | Quality | Clear, colorless liquid; | ND |
| | | practically free of particulates | |

¹Non-GMP laboratory-scale drug lots meet or exceed GMP specifications, demonstrating a robust manufacturing method.
²Laboratory-scale drug lots manufactured according to the specification.

### Example 12

### Exemplary OAS Protein Drug GMP Release Specification 2

| | | |
|---|---|---|
| Method | Attribute | Acceptance Criteria |

Release Assays reported on C of A

| | | |
|---|---|---|
| Appearance | Quality | Colourless to slightly yellow liquid: essentially |
| | | free of particulates |
| pH | Quality | 6.4 ± 0.5 (at 25° C.) |
| Osmolality | Purity | 788 ± 100 mOsm/kg |
| Drug Concentration by A280 | Strength | 10-13.5 mg/ml |
| Activity Assay | Potency | 50 to 150% of reference standard activity |
| SDS-PAGE reduced | Identity | Conforms to reference standard: main band at |
| | | approximately 38 kDa |
| SDS-PAGE non-reduced | Identity | Conforms to reference standard: main band at |
| | | approximately 38 kDa |
| Reversed phase HPLC | Purity | ≧80% main peak |
| Size exclusion HPLC | Purity | ≧95% main peak |
| IEX-HPLC | Purity | ≧80% main peak |
| Residual host cell protein | Impurity | ≦100 ng/mg |
| Endotoxin | Safety | ≦1.0 EU/mg |
| Bioburden | Safety | <10 CFU/mL |
| Residual host cell DNA | Impurity | Report |

Information Assays reported on Analytical Report

| | | |
|---|---|---|
| Residual kanamycin | Impurity | Report |
| Residual IPTG | Impurity | Report |
| Residual Antifoam | Impurity | Report |

Characterisation Assays reported on Technical Reports

| | | |
|---|---|---|
| Western blot | Identity | Conforms to reference standard: main band at |
| | | approximately 38 kDa. Migration of main band |
| | | and presence of product related bands |
| | | comparable to reference standard |
| N terminal sequencing | Identity | Conforms to reference standard. Primary |
| | | sequence: |
| | | Met, Asp, Leu, Arg, Asn, Thr, Pro, Ala, Lys, |
| | | Ser (MDLRNTPAKS) |
| SDS-PAGE reduced, silver stain | Identity/ | Conforms to reference standard: main band at |
| | characterisation | approximately 38 kDa |
| Intact mass by ESI-MS | Identity | Conforms to reference standard 39728 ± 8 Da |

### Example 13

### Analytical Testing of Drug (OAS Protein API) Lots Produced by Process at 8-100L Scale

| Analytical | Run 1 (~8L | Run 2 (~8L | | |
|---|---|---|---|---|
| method | Scale) | Scale) | Run 3 (~100L Scale) | Run 4 (~100L Scale) |
| A280 | 6.2 mg/ml | 10.8 mg/ml | 10.3 mg/ml | 10.6 mg/ml |
| measurement | | | | |
| SDS-PAGE | Main band | Main band | Main band migrates in the | Main band migrates in the |
| reduced | migrates in the | migrates in the | same position as the | same position as the |
| | same position | same position | reference standard. | reference standard. |
| | as the reference | as the reference | Comparable to reference | Comparable to reference |
| | standard. | standard. | standard | standard |
| | Comparable to | Comparable to | | |
| | reference | reference | | |
| | standard | standard | | |
| SDS-PAGE | Main band | Main band | Main band migrates in the | Main band migrates in the |
| non-reduced | migrates in the | migrates in the | same position as the | same position as the |
| | same position | same position | reference standard. | reference standard. |
| | as the reference | as the reference | Comparable to reference | Comparable to reference |
| | standard. | standard. | standard | standard |
| | Comparable to | Comparable to | | |
| | reference | reference | | |
| | standard | standard | | |
| OAS activity by | Kcat value = 48 | Kcat value = 57 | Kcat value = 42 | Kcat value = 35 |
| HPLC Assay | (Ref std PT63 | (Ref std PT63 | (Ref std PT63 Kcat value = | (Ref std PT63 Kcat value = |
| | Kcat value = | Kcat value = | 34) | 37) |
| | 54) | 60) | | |
| Appearance | Not tested | Not tested | Colourless clear liquid, | Colourless clear liquid, |
| | | | practically free of | practically free of |
| | | | particulate matter | particulate matter |
| Reverse phase | 76.9% main | 75.9% main | 85.6% main peak | 86.9% main peak |
| HPLC purity | peak | peak | | |
| Ion exchange | 82.0% main | 79.6% main | 87.3% main peak | 88.1% main peak |
| HPLC purity | peak | peak | | |
| pH at 25° C. | Not tested | Not tested | pH 6.5 (at 25° C.) | pH 6.6 (at 25° C.) |
| Endotoxin | 0.2 EU/mg | 10.2 EU/mg | 0.65 EU/mg | 0.3 EU/mg |
| (turbidimetric | | | | |
| LAL test) | | | | |
| Bioburden | Not tested | Not tested | <10 FCU/ml Total aerobic | <10 FCU/ml Total aerobic |
| (Microbial limit | | | microbial count. <10 CFU/ml | microbial count. <10 CFU/ml |
| test) | | | for combined | for combined |
| | | | yeasts & moulds. | yeasts & moulds. |
| | | | Salmonella and E. coli | Salmonella and E. coli |
| | | | species absent by test. | species absent by test. |
| | | | S. Aureus and P. aeruginosa | S. Aureus and P. aeruginosa |
| | | | absent by test. Absence of | absent by test. Absence of |
| | | | inhibition/enhancement | inhibition/enhancement |
| Host Cell DNA | <78.1 pg/ml | <78.1 pg/ml | 275.8 ± 52 pg/ml | <78.1 pg/ml |
| (Q-PCR) | | | (= 27 pg/mg) | |
| Host cell protein | 38 ng/mg | 40 ng/mg | 12 ng/mg | 9 ng/mg |
| (ELISA) | | | | |
| Osmolality | Not tested | Not tested | 789 mOsm/kg | 787 mOsm/kg |
| Size exclusion | 97.5% main | 98.2% main | 99.4% main peak | 98.5% main peak |
| HPLC—purity | peak | peak | | |
| Kanamycin | Not tested | Not tested | <200 ng/mg | <200 ng/mg |
| (LC/MS) | | | | |
| IPTG (GC/MS) | Not tested | Not tested | <30 ng/mg | <30 ng/mg |
| Residual anti- | Not tested | Not tested | Not tested | Not tested |
| foam | | | | |
| Western Blot | Not tested | Not tested | Conforms. Migration of | Conforms. Migration of |
| analysis | | | main band and presence of | main band and presence of |
| | | | product related bands | product related bands |
| | | | comparable to reference | comparable to reference |
| | | | standard | standard |
| N-terminal | Not tested | Not tested | Conforms to reference | Conforms to reference |
| sequence | | | standard. Primary sequence: | standard. Primary sequence: |
| analysis (10 | | | Met, Asp, Leu, Arg, Asn, | Met, Asp, Leu, Arg, Asn, |
| cycles) | | | Thr, Pro, Ala, Lys, Ser | Thr, Pro, Ala, Lys, Ser |
| | | | (MDLRNTPAKS) | (MDLRNTPAKS) |
| SDS-PAGE | Not tested | Not tested | Not tested | Not tested |
| (reduced) | | | | |
| Silver stained | | | | |
| Intact mass by | 39,733 Da | 39,732 Da | 39,729 Da | 39,730 Da |
| MS | | | | |

### Example 14

### Exemplary Intravenous Formulation 2

The OAS proteins were formulated in a buffered solution suitable for intravenous administration to a mammal that included the following components: 10.8 mg/mL OAS protein, 10 mM sodium citrate, 270 mM sodium chloride, 7% sucrose, pH 6.4.

The foregoing specification, including the specific embodiments and examples, is intended to be illustrative of the present invention and is not to be taken as limiting. Numerous other variations and modifications can be effected without departing from the true spirit and scope of the invention. All patents, patent publications, and non-patent publications cited are incorporated by reference herein.

1. A method for producing oligoadenylate synthetase (OAS) proteins comprising:
a) culturing a host cell containing an expression vector in growth medium under conditions wherein the OAS protein is expressed;
b) recovering the host cell from the growth medium; and
c) isolating the OAS protein from the host cell.

2. The method of claim 1 wherein said OAS protein is selected from the group consisting of SEQ ID NO:9-16.

3. The method of claim 1 wherein said expression vector comprises a polypeptide selected from the group consisting of SEQ ID NO:1-8.

4. The method of claim 1 wherein said host cell is E. coli.

5. The method of claim 1 further comprising a step of assessing in-process protein purity.

6. The method of claim 5 wherein said step comprises an enzyme-linked immunosorbent assay.

7. The method of claim 5 wherein said step comprises Limulus Amoebocyte Lysate endotoxin assay.

8. The method of claim 1 further comprising a step of assessing final purified protein purity.

9. A method of purifying an OAS protein comprising:
a) culturing a host cell containing an expression vector in growth medium under conditions wherein the OAS protein is expressed;
b) recovering the host cell from the medium;
c) isolating the OAS protein from the host cell; and
d) purifying the OAS protein using affinity chromatography.

10. The method of claim 9 wherein said OAS protein comprises an affinity tag.

11. The method of claim 9 wherein said OAS protein is selected from the group consisting of SEQ ID NO:9-16.

12. The method of claim 9 wherein said expression vector comprises a polypeptide selected from the group consisting of SEQ ID NO:1-8.

13. The method of claim 9 wherein said host cell is E. coli.

14. A method of purifying an OAS protein comprising:
a) culturing a host cell containing an expression vector in growth medium under conditions wherein the OAS protein is expressed;
b) recovering the host cell from the medium;
c) isolating the OAS protein from the host cell; and
d) purifying the OAS protein using anion exchange chromatography.

15. The method of claim 14 wherein said OAS protein comprises an affinity tag.

16. The method of claim 14 wherein said OAS protein is selected from the group consisting of SEQ ID NO:9-16.

17. The method of claim 14 wherein said expression vector comprises a polypeptide selected from the group consisting of SEQ ID NO:1-8.

18. The method of claim 14 wherein said host cell is E. coli.


SEQUENCE LISTING (16 sequences)

SEQ ID NO: 1 - DNA, 1038 bp, Homo sapiens
atggatttgcgtaacaccccagcaaaaagccttgataaatttattgaagattatttactgcctgatacttgttttcgcatgcaaatcaaccatgcaatcgatatcatttgtggatttttaaaagaacgttgctttcgtggctcgtcatacccagtctgtgttagcaaagtcgtcaaaggcggttcctcaggaaaaggcaccaccctccgcggtcgttcagatgccgatttagtcgtttttttatcccctcttacaacgttccaagatcaacttaatcgtcgtggtgaatttattcaagaaattcgtcgtcaattggaagcatgtcaacgcgagcgcgccttttctgtcaaatttgaagtacaagcaccgcgttggggtaatcctcgtgccctctcatttgttttaagctctttacaattaggcgaaggcgtggaatttgatgtcctccctgcatttgatgcacttggtcagttaaccggtggctacaaacctaaccctcagatctatgtgaaattaatcgaagaatgtaccgacctgcagaaagaaggagaatttagcacctgctttacggaactgcaacgtgatttcctcaaacaacgtcccactaaactcaaatcattgattcgtcttgttaaacactggtatcaaaattgcaaaaaaaaacttggaaaactgcccccacaatacgcactggaacttcttaccgtatatgcatgggaacgtggctctatgaaaacgcattttaataccgcccaaggttttcgtactgtattagaattagtaatcaactatcaacaactgtgtatctattggaccaaatactatgattttaaaaaccctattatcgaaaaatatttacgtcgtcagcttaccaaacctcgtcctgtaattttagacccggcagaccctacaggtaatcttggtggcggtgatccaaaaggctggcgccaactcgctcaggaagcagaagcgtggctcaattatccttgttttaaaaattgggacggctccccggtcagctcgtggattttactgtaa

SEQ ID NO: 2 - DNA, 1038 bp, Homo sapiens
atggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgtga

SEQ ID NO: 3 - DNA, 1038 bp, Homo sapiens
atggatttgcgtaacaccccagcaaaaagccttgataaatttattgaagattatttactgcctgatacttcttttcgcatgcaaatcaaccatgcaatcgatatcatttccggatttttaaaagaacgttgctttcgtggctcgtcatacccagtctctgttagcaaagtcgtcaaaggcggttcctcaggaaaaggcaccaccctccgcggtcgttcagatgccgatttagtcgtttttttatcccctcttacaacgttccaagatcaacttaatcgtcgtggtgaatttattcaagaaattcgtcgtcaattggaagcatgtcaacgcgagcgcgccttttctgtcaaatttgaagtacaagcaccgcgttggggtaatcctcgtgccctctcatttgttttaagctctttacaattaggcgaaggcgtggaatttgatgtcctccctgcatttgatgcacttggtcagttaaccggtggctacaaacctaaccctcagatctatgtgaaattaatcgaagaatgtaccgacctgcagaaagaaggagaatttagcacctgctttacggaactgcaacgtgatttcctcaaacaacgtcccactaaactcaaatcattgattcgtcttgttaaacactggtatcaaaattgcaaaaaaaaacttggaaaactgcccccacaatacgcactggaacttcttaccgtatatgcatgggaacgtggctctatgaaaacgcattttaataccgcccaaggttttcgtactgtattagaattagtaatcaactatcaacaactgtgtatctattggaccaaatactatgattttaaaaaccctattatcgaaaaatatttacgtcgtcagcttaccaaacctcgtcctgtaattttagacccggcagaccctacaggtaatcttggtggcggtgatccaaaaggctggcgccaactcgctcaggaagcagaagcgtggctcaattatccttgttttaaaaattgggacggctccccggtcagctcgtggattttactgtaa

SEQ ID NO: 4 - DNA, 1095 bp, Homo sapiens
atgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggtgagacctcctgcttcctccctgccattcatccctgcccctctccatgaagcttga

SEQ ID NO: 5 - DNA, 1203 bp, Homo sapiens
atgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtgcagacgatgagaccgacgatcccaggaggtatcagaaatatggttacattggaacacatgagtaccctcatttctctcatagacccagcacactccaggcagcatccaccccacaggcagaagaggactggacctgcaccatcctctga

SEQ ID NO: 6 - DNA, 1380 bp, Homo sapiens
atgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgctgaaagcaacagtacagacgatgagaccgacgatcccaggacgtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagargacaaagctcctcagtgagctggtgtataatccaggacagaacccaggtctcctgactcctggccttctatgccctctatcctatcatagataa

SEQ ID NO: 7 - DNA, 1155 bp, Homo sapiens
atgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgatgagacaaaggctcagagaggtgaggtcacttgctcaaggacatcagctaacaagtggtggaaatggaattcaagctcagtggactctaaagccagtgctcatgtcactgtgctaa

SEQ ID NO: 8 - DNA, 1244 bp, Homo sapiens
atgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaagacaaagctcctcagtgagctggtgtataatccaggacagaacccaggtctctga

SEQ ID NO: 9 - protein, 346 aa, Homo sapiens
  VARIANT 1: Xaa = Met or -Met (Met deleted)
XMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILX

SEQ ID NO: 10 - protein, 346 aa, Homo sapiens
  VARIANT 1: Xaa = Met OR -Met (Met deleted)
XMDLRNTPAKSLDKFIEDYLLPDTXFRMQIXHAIDIIXGFLKERCFRGSSYPVXVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILX

SEQ ID NO: 11 - protein, 364 aa, Homo sapiens
  VARIANT 31: Xaa = Asp OR Asn
MMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILLVRPPASSLPFIPAPLHEA

SEQ ID NO: 12 - protein, 400 aa, Homo sapiens
  VARIANT 31: Xaa = Asp OR Asn
MMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILLAESNSXDDETDDPRXYQKYGYIGTHEYPHFSHRPSTLQAASTPQAEEDWTCTIL

SEQ ID NO: 13 - protein, 384 aa, Homo sapiens
  VARIANT 31: Xaa = Asp OR Asn
MMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILLMRQRLREVRSLAQGHQLTSGGNGIQAQWTLKPVLMSLC

SEQ ID NO: 14 - protein, 459 aa, Homo sapiens
  VARIANT 31: Xaa = Asp OR Asn
MMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILLLKATVQTMRPTIPGXIRNMVTLEHMSTLISLIDPAHSRQHPPHRQKRTGPAPSSECQCILGERAPVLSGPVPSFSGGTLDPEXTKLLSELVYNPGQNPGLLTPGLLCPLSYHR

SEQ ID NO: 15 - protein, 414 aa, Homo sapiens
  VARIANT 31: Xaa = Asp OR Asn
MMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKNWDGSPVSSWILLTQHTPGSIHPTGRRGLDLHHPLNASASWGKGLQCYLDQFLHFQVGLLIQRXQSSSVSWCIIQDRTQVS

SEQ ID NO: 16 - protein, 334 aa, Homo sapiens
  VARIANT 1: Xaa = Met or -Met (Met deleted)
XMDLRNTPAKSLDKFIEDYLLPDTCFRMQIXHAIDIICGFLKERCFRGSSYPVCVSKVVKGGSSGKGTTLRGRSDADLVVFLSPLTTFQDQLNRRGEFIQEIRRQLEACQRERAXSVKFEVQAPRWXNPRALSFVLSSLQLGEGVEFDVLPAFDALGQLTGXYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKXPIIEKYLRRQLTKPRPVILDPADPTGNLGGGDPKGWRQLAQEAEAWLNYXCFKN